(19) United States
(12) Patent Application Publication
(10) Pub. No.: US 2004/0014052 A1
(43) Pub. Date: Jan. 22, 2004

(54) Process for regulating gene expression

(76) Inventors: Warren H. Kurtz, Lyndhurst, NJ (US); Stuart G. MacDonald, Pultneyville, NY (US); Howard J. Greenwald, Rochester, NY (US)

Correspondence Address: HOWARD J. GREENWALD P.C., 349 W. COMMERCIAL STREET SUITE 2490, EAST ROCHESTER, NY, 14445-2408, US

(21) Appl. No.: 10/200,980

(22) Filed: Jul. 22, 2002

(51) Int. Cl. C12Q 1/68; C12M 1/34; C12N 15/85; C12P 21/06

(52) U.S. Cl. 435/6; 435/287.2; 435/455; 435/69.1

(57) ABSTRACT

A process for stimulating the expression of a gene in which two different DNA constructs are disposed within a biological system and contacted with electromagnetic radiation, thereby causing each of the DNA constructs to express a gene product.

### FIELD OF THE INVENTION

A process for regulating gene expression in a biological system in which at least one electromagnetic signal is used to cause upregulation of a first gene product and a second gene product, each of which contains an electromagnetic response element.

### BACKGROUND OF THE INVENTION

It is known that certain nucleotide sequences in certain constructs, upon being stimulated by electromagnetic fields, tend to cause the expression of protein. During the last 20 years, there have been many publications disclosing the effects of electromagnetic fields upon protein synthesis.

Thus, in 1986, Reba Goodman and A. S. Henderson, in an article entitled “Sine Waves Enhance Cellular Transcription” (appearing in Bioelectromagnetics, 7, 23-29, 1986), showed that RNA levels can be increased by electromagnetic fields ranging in frequency from 15 to 4,400 Hertz and that the RNA levels can be so enhanced by a factor of ten or more. For a discussion of this and related articles, reference may be had to U.S. Pat. No. 6,263,878.

In 1988, Reba Goodman and A. S. Henderson, in an article appearing in the Proceedings of the National Academy of Science, U.S.A. (at 85, 3298-3932), demonstrated changes in the transcription and translation stages of protein synthesis following exposure to weak electromagnetic fields.

It was known as early as 1989 that electromagnetic fields can affect biological processes in ways that are beneficial. Reference may be had, e.g., to page 4 of “Electromagnetic Fields: Biological Interactions and Mechanisms,” edited by Martin Blank (American Chemical Society, Washington, D.C., 1995) and to the footnotes 8 and 9 appearing at page 9 of such book.

In 1994, in an article by Reba Goodman and others (“Increased levels of HSP70 transcripts induced when cells are exposed to low level electromagnetic fields,” appearing in Bioelectrochemistry and Bioenergetics 1994; 33: 115-120), it was demonstrated that low level electromagnetic fields activate heat shock factor.

Also in 1994, in an article entitled “Specific Region of the c-myc Promoter is Responsive to Electric and Magnetic Fields,” Han Lin, Reba Goodman, and A. S. Henderson disclosed the isolation of a region in the promoter of the c-myc gene that was responsive to electromagnetic radiation such that expression of the gene was upregulated.

In 1996, in U.S. Pat. No. 5,566,685 (see Column 2), it was disclosed that: “At the present time overwhelming evidence exists which shows that a wide range of biological effects are possible even at very low levels of exposure . . . . These effects include changes in transcription of specific genes, changes in enzyme activities, production of morphological abnormalities and biochemical modifications in developing chick embryos, stimulation of bone cell growth, suppression of nocturnal melatonin in humans, and alteration in cellular calcium pools. [Goodman, R. L. . . . ‘Exposure of human cells to low-frequency electromagnetic fields results in quantitative changes in transcripts,’ Biochim. Biophys. Acta, 1009:216-220, 1989 . . . . ”

In 1998, in International patent publication WO 98/37907(at pages 6-7 thereof), it was disclosed that: “In 1988 Blank and Goodman reported an analysis of the synthesis of proteins induced by electromagnetic fields in salivary glands of Sciara. They reported that the protein synthesis modifications caused by heat shock (HS) and by electromagnetic (EM) stimulation had certain similarities. However, they noted a significant difference in the effects of HS and EM stimulation on protein synthesis. They found that electromagnetic (EM) fields caused an increased synthesis of proteins in the molecular weight range 20-50 kD but they discovered a generally reduced synthesis of polypeptides in the 50-90 kD molecular weight range . . . . Blank and Goodman report that the synthesis of HSP70 is either reduced by EM fields or only a negligible (2%) increase occurs under certain signal conditions.” For a similar disclosure, reference may also be had to U.S. Pat. No. 5,968,527.

In 2001, it was again disclosed that certain nucleotide sequences in certain constructs, upon being stimulated by electromagnetic fields, tend to cause the expression of protein. In an article entitled “Regulating Genes with Electromagnetic Response Elements,” by Hana Lin et al. (Journal of Cellular Biochemistry 81:143-148 (2001), it was disclosed that a 900 base pair segment of the c-myc promoter is required for the induction of c-myc expression by electromagnetic fields. As disclosed in this article (see page 145), the transfectants produced by the process of the article were exposed to an 8 microTesla field for 30 minutes (at 60 Hertz) followed by an additional 30 minutes out of the field prior to protein extraction for the luciferase assay. “Luciferase activity increased at an average of 61%.”

In 2002, in United States published patent application US2002/0072646, it was disclosed that “A new method for inducing the stress response has been found. Goodman and Blank report induction of the stress response via exposing tissue to low energy magnetic fields. The methods described involve exposing tissue to specific alternating current electromagnetic fields. They used various magnetic field parameters under 1 Gauss and less than 300 Hertz . . . . In comparison to thermal stress . . . , magnetic fields induce similar stress responses at energies which are fourteen orders of magnitude lower.” (See page 1 of the published patent application.)

However, it should be noted that there are several prior art teachings indicating that the increase in protein production in response to an electromagnetic field(s) is often only transient. Thus, as is disclosed in another paper by Li Han et al., entitled “Application of Magnetic Field—Induced Heat Shock Protein 70 for Presurgical Cytoprotection” (Journal of Cellular Biochemistry 71:577-583, 1998), it was disclosed that the production of heat shock protein 70 decreased from about 40 percent at 20 minutes to about 20% at 180 minutes (see FIG. 3 at page 580) and became refractory to further stimulation, no longer yielding an increase in protein production upon such stimulation. At page 580 of this article, it was disclosed that: “Cells in a refractory state, initially stimulated by magnetic fields, could not be restimulated by heat shock.”

Because, in the Lin et al. process, the cells become refractory and cannot be restimulated, such process cannot be used to reliably and durably produce protein over a relatively long period of time at a high yield. Furthermore, because such cells become refractory, they cannot be used to reliably and durably treat an organism by stimulation of such cells over a long a period of time.

It is an object of this invention to provide a process for increasing the expression of a specified gene in which the protein production does not substantially decrease in a short period of time.

It is another object of this invention to provide a process for controlling genetic activity with a construct that is responsive to electromagnetic radiation.

It is yet another object of this invention to provide a process for effectively selecting control elements for use in specific cell types and for effecting specific cellular behavior(s) in response to electromagnetic radiation.

It is another object of this invention to provide apparatuses and methods for using cell cultures in manufacturing processes wherein the behavior of the cell population can be influenced in order to optimize productive output.

### SUMMARY OF THE INVENTION

In accordance with this invention, there is provided a process for stimulating the expression of a gene comprising the steps of disposing a first electromagnetic responsive DNA construct in a biological system, disposing a second electromagnetic responsive DNA construct in such biological system, stimulating said first electromagnetic responsive DNA construct with a electromagnetic energy, stimulating said second electromagnetic responsive DNA construct with electromagnetic energy, expressing a first RNA transcript from said first electromagnetic responsive DNA constuct, and expressing a second RNA transcript from said second electromagnetic responsive DNA construct.

### BRIEF DESCRIPTION OF THE DRAWINGS

The invention will be described by reference to the following drawings, in which like numerals refer to like elements, and in which:

FIG. 1 is a flow diagram of one preferred process;

FIG. 2 is a schematic of a transfected biological system used in the process of FIG. 1;.

FIG. 3 is a flow diagram of a process for determining nucleotide sequences that, in response to electromagnetic radiation, promote expression of a gene;

FIG. 4 illustrates a particular construct that can be made by the process of FIG. 3;

FIG. 5 is a flow chart of a process for determining what positive regulatory control factors are associated with any particular promoter 22;

FIG. 6 is a schematic of a linked construct that may be used in the process of FIG. 5;

FIG. 7 is a schematic of a transfection process for targeting specific genes in a living organism with the active region(s) of the promoter 22;

FIG. 8 is a schematic of a process for creating a living organism with a multiplicity of naturally occurring gene assemblies responsive to different electromagnetic radiations;

FIG. 9 a flow diagram of a process for identifying synthetic promoters 22 that will increase gene expression;

FIG. 10 illustrates one preferred process for producing a desired protein that can be used ex vivo;

FIG. 11 is a schematic diagram of a self-regulating reactor vessel 190 for the optimum production of a desired gene product;

FIG. 12 is a schematic of a process for trans-regulation, or cross-regulation of gene products using electromagnetic response elements and an externally applied electromagnetic field;

Each of FIGS. 13, 14, 15, 16, and 17 are schematics of specified bioreactor systems;

FIG. 18 is a functional diagram showing the various control methods used in the bioreactor systems depicted in FIGS. 13 through 17;

FIG. 19 is a graph representing the relationship between cell population and production of desired bioreactor products under nominal conditions, subnominal conditions, and under superoptimal conditions enabled by the practice of this invention;

FIG. 20 is a flow diagram of another preferred process of the invention for minimizing a refractory response to electromagnetic radiation;

FIG. 21 is schematic diagram of a process for producing a DNA vaccine;

FIG. 22 is a schematic diagram of a biological assembly comprised of DNA constructs with electromagnetic response elements and implantable means for stimulating such constructs;

FIG. 23 is a flow diagram of a process for minimizing (“downregulating”) the production of an undesired gene product by causing the production of a negative transcription factor or a negative translation factor for such product with the use of electromagnetic radiation; and

FIG. 24 is a flow diagram of a process for determining the unknown function of a protein by inhibiting its production with the use of electromagnetic radiation to stimulate the expression of a negative control factor.

### DESCRIPTION OF THE PREFERRED EMBODIMENTS

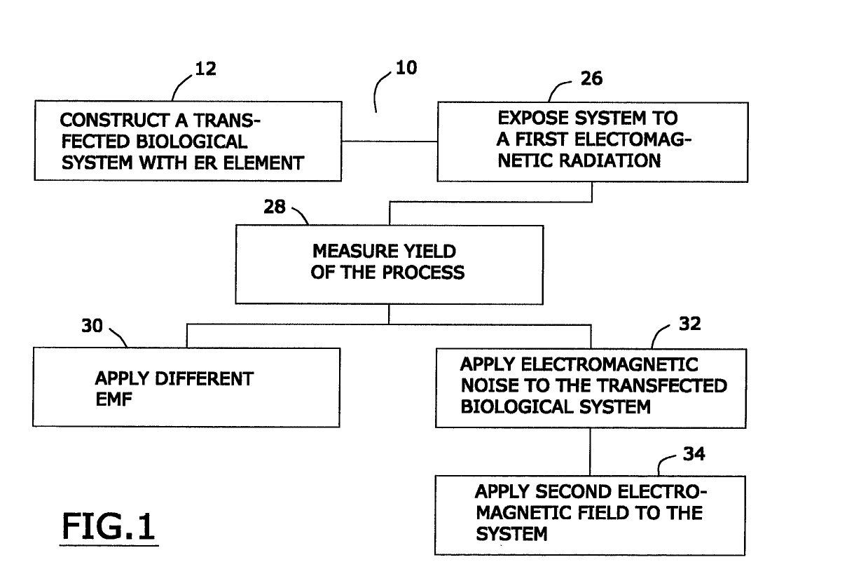

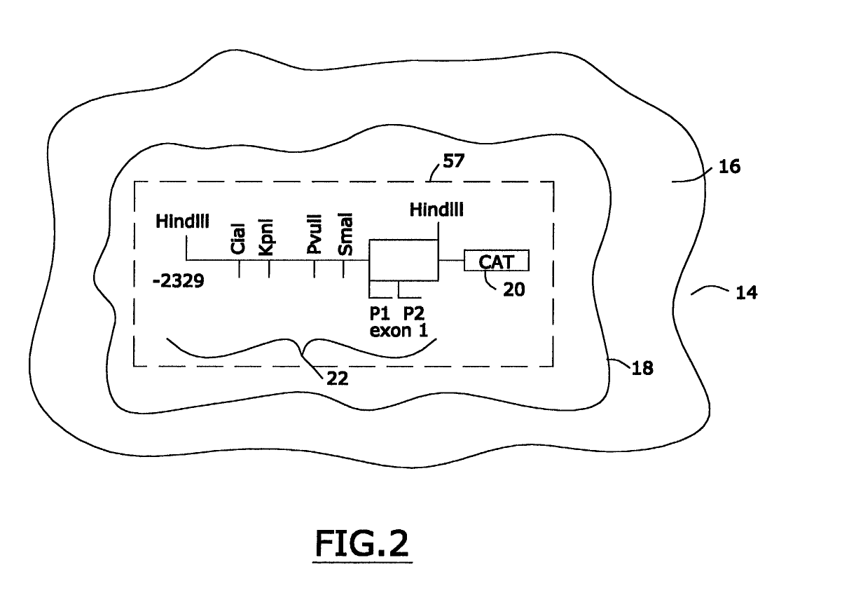

FIG. 1 is a flow diagram of one preferred process 10 of this invention. In step 12 of this process, a transfected biological system with an electromagnetic response (ER) element is constructed. FIG. 2 is a schematic of such transfected biological system. Each of these Figures will be discussed in greater detail elsewhere in this specification.

Referring to FIG. 2, and to the preferred embodiment depicted therein, it will be seen that host cell 14 is comprised of cytoplasm 16 and, disposed therein, nucleus 18. As will be apparent, the cell 14 depicted in FIG. 2 is a eurkaryotic cell. Prokaryotic cells also may be used in the process of this invention.

Referring again to FIG. 2, and in the preferred embodiment depicted therein, disposed within nucleus 18 is a structural gene 20 that, in the embodiment depicted, is a CAT (chloramphenicol acetyl-transferase) gene. As will be apparent, structural gene 20 can be any other gene.

In the process of this invention, the gene 20 preferably is a polynucleotide DNA (deoxynucleic acid) sequence that codes for and produces a messenger RNA (ribonucleic acid) sequence. As is known to those skilled in the art, a messenger RNA is a single-stranded molecule that is synthesized during transcription, that is complementary to one of the strands of double-stranded DNA, and that serves to transmit the genetic information contained in DNA to the ribosomes for protein synthesis. See, e.g., page 295 of J. Stenesh's “Dictionary of Biochemsitry and Molecular Biology,” Second Edition (John Wiley & Sons, New York, N.Y., 1989).

The messenger RNA may produce protein by the process of translation. As is known to those skilled in the art (see, e.g., page 491 of the aforementioned Stenesh reference), translation is the process in which the genetic information of the messenger RNA is used to specify and direct the synthesis of a polypeptide chain on a ribosome.

The messenger RNA is not always used to produce protein. When antisense messenger RNA is expressed, the translation process does not take place to produce the desired, functional protein. Antisense messenger RNA, its properties, and its uses, are all well known. Reference may be had, e.g., to U.S. Pat. Nos. 6,410,723, 6,245,512, 5,998,194, 5,916,763, 6,388,113, 6,254,890, 6,043,409, 5,955,269, and the like.

Referring again to FIG. 2, and in one preferred embodiment, gene 20 preferably is a structural gene. A structural gene is a gene, the nucleotide sequence of which determines the amino acid sequence of a polypeptide chain.

Referring again to FIG. 2, structural gene 20 is linked (covalently ligated) to a promoter 22; the promoter 22 is a promoter that responds to electromagnetic radiation and preferably causes transcription of the gene 20.

One may use any promoter 22 that causes transcription of a gene in response to electromagnetic radiation. In the embodiment depicted in FIG. 2, such promoter is the c-myc promoter. This promoter is well known in the art. Reference may be had, e.g., to U.S. Pat. No. 6,140,052 (cMYC is regulated by Tcf-4), U.S. Pat. Nos. 6,037,329, 5,972,643, 5,807,884, 5,795,975, and the like. The entire disclosure of each of these United States patents is hereby incorporated by reference into this specification.

By way of further illustration, one may also use the HSP (heat shock protein) 70 promoter. The HSP-70 promoter also is well known; reference may be had, e.g., to U.S. Pat. Nos. 6,403,783, 6,225,121, 6,130,074, 6,046,025, 5,965,393, and the like. The entire disclosure of each of these United States patents is hereby incorporated by reference into this specification.

A substantial amount of literature has been published regarding the response of these promoters 22 to electromagnetic radiation. Reference may be had, e.g., to an article by Hana Lin et al. “Specific Region of the c-myc Promoter is Responsive to Electric and Magnetic Fields” (Journal of Cellular Biochemistry 54:281-288, 1994), to an article by Hana Lin et al. entitled “Electromagnetic Field Exposure Induces Rapid, Transitory Heat Shock Factor Activation in Human Cells (Journal of Cellular Biochemistry 66:482-488, 1997), to an article by Hana Lin et al. entitled “Myc-Mediated Transactivation of HSP70 Expression Following Exposure to Magnetic Fields” (Journal of Cellular Biochemistry 69:181-188(1998), to an article by H. Lin et al. entitled “A Magnetic Field-Responsive Domain in the Human HSP70 Promoter” (Journal of Cellular Biochemistry 75:170-176, 1999), to an article by Hana Lin et al. entitled “Regulating Genes with Electromagnetic Response Elements” (Journal of Cellular Biochemistry, 81:143-148, 2001), and the like.

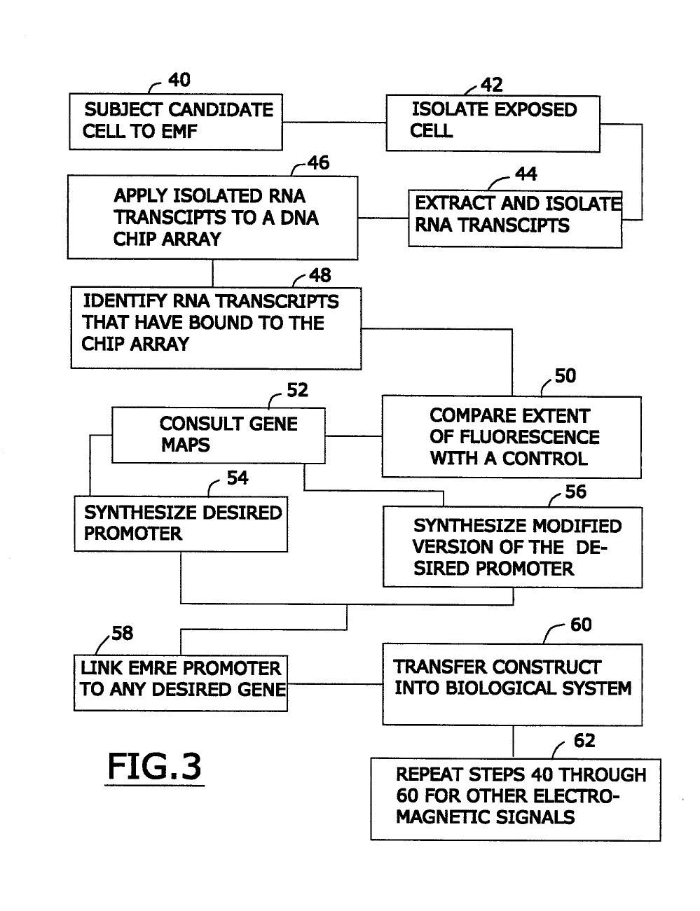

The aforementioned articles discuss two of the promoters 22 that may be used, the HSP70 promoter that promotes the expression of heat shock protein 70 (or any gene that is linked to such promoter), and the c-myc promoter. One may determine the existence of other promoters that cause the expression of protein upon stimulation by electromagnetic radiation by the process depicted in FIG. 3. Such promoter or promoters can be isolated and cloned.

Referring to FIG. 3, and in step 40 thereof, a candidate cell suspected of containing the promoter 22 is subjected to electromagnetic radiation (EMF). It is preferred in step 40 to subject such cell to electromagnetic radiation for less than about 1 hour. It is more preferred to subject such cell to electromagnetic radiation for less than about 30 minutes.

The cell so subjected to the electromagnetic radiation preferably is disposed within a biological system, such as a biological organism, a cell culture, etc. These cells may be in vivo, or they may be ex vivo.

After the cell has been exposed to electromagnetic radiation in step 40, in step 42 the exposed cell is isolated by well-known conventional techniques. Thereafter, in step 44 of the process, the MRNA (messenger ribonucleic acid) transcripts from the cell are extracted and isolated.

The RNA transcripts isolated in step 44 are first preferably converted into fluorescent gene probes and then applied to a DNA (deoxyribonucleic acid) chip array by conventional means in step 46. Reference may be had, e.g., to U.S. Pat. No. 5,547,860. The entire disclosure of this United States patent is hereby incorporated by reference into this specification.

These DNA chip arrays are well known and are described, e.g. in an article appearing in the Jun. 24, 2002 edition of U.S. News and World Report (see page 52 thereof). Reference also may be had, e.g., to U.S. Pat. Nos. 6,399,311, 6,142,681, 6,136,541, 5,843,651, 5,599,668, and the like. The entire disclosure of each of these United States patents is hereby incorporated by reference into this specification.

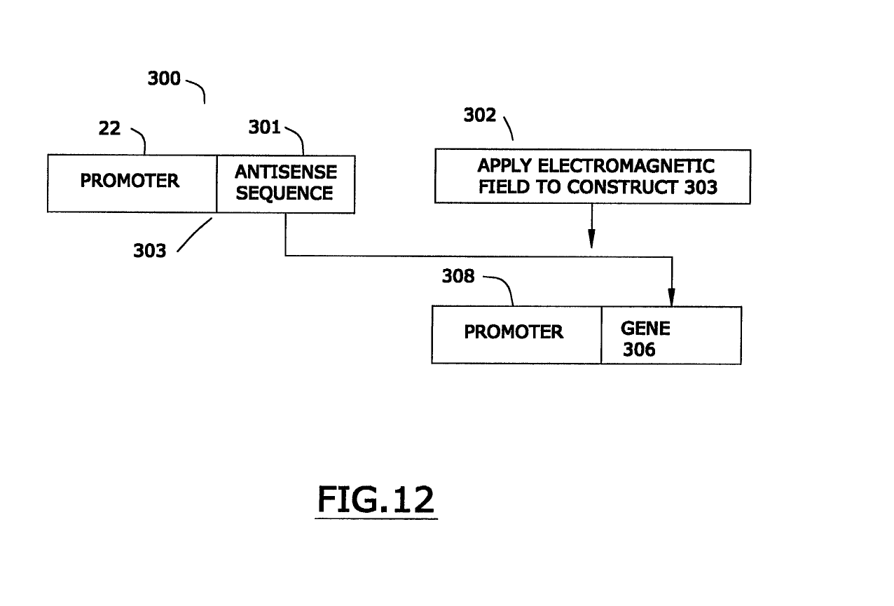

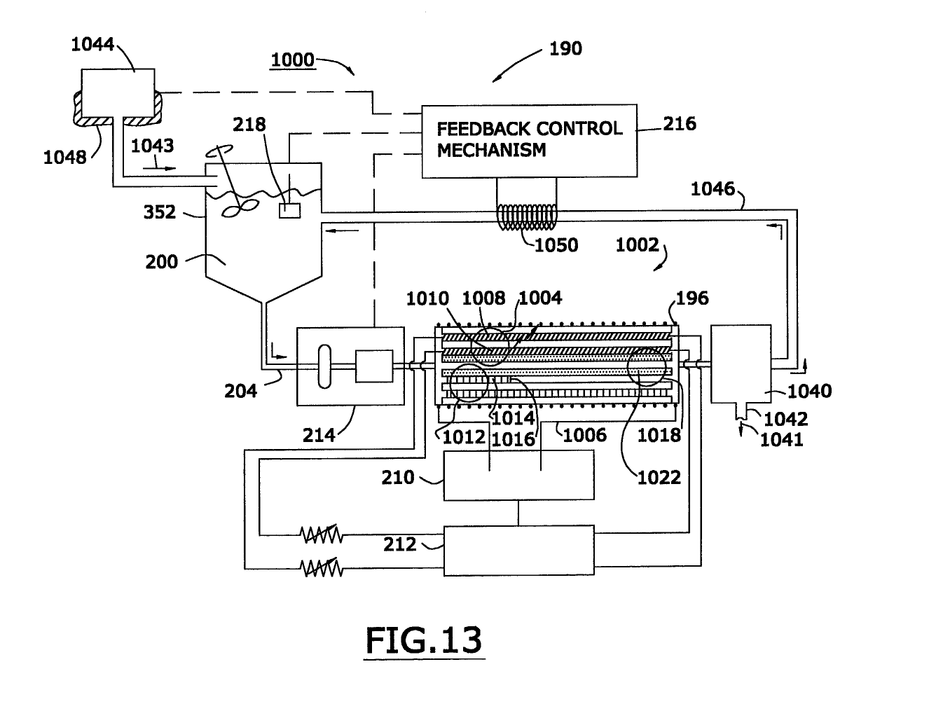

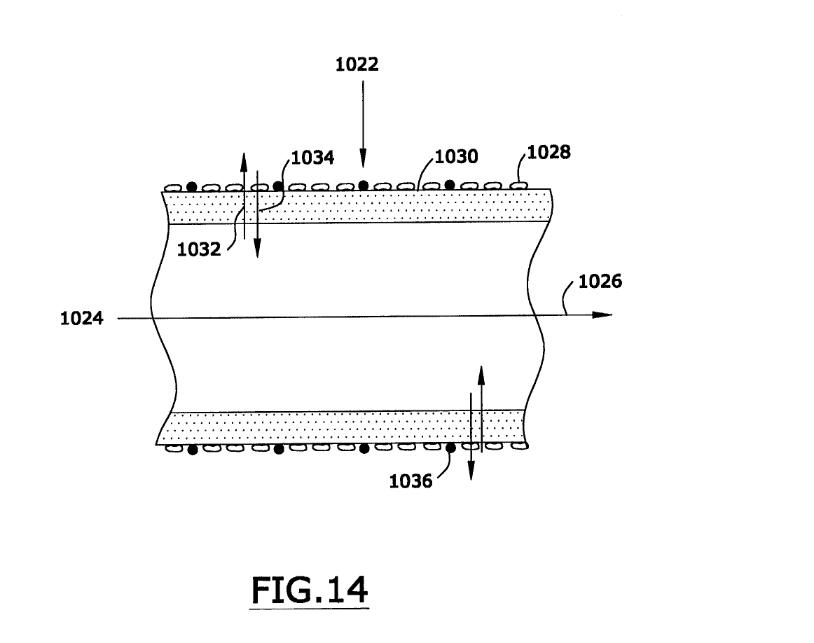

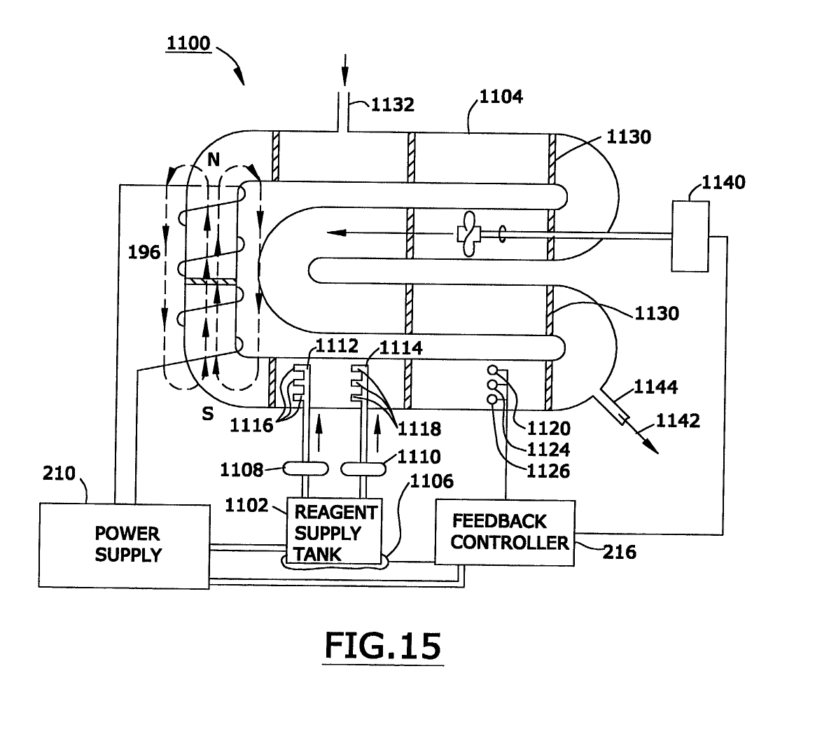

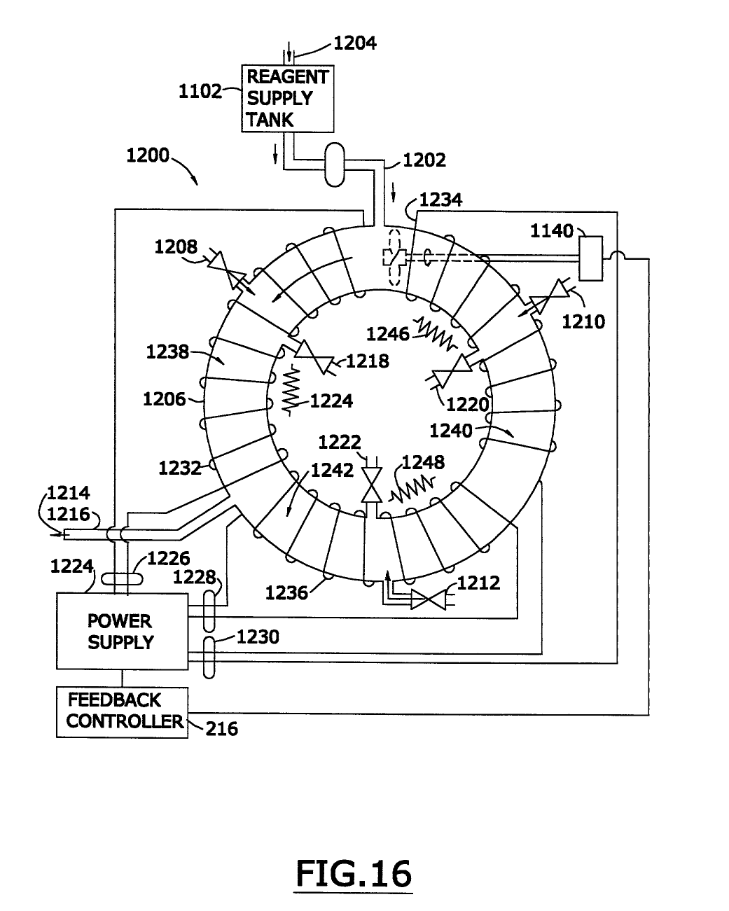

By way of yet further illustration, and as is described in U.S. Pat. No. 6,407,858 (the entire disclosure of which is hereby incorporated by reference in to this specification), one may use ordered arrays of biological material such as biochips. In one case an ordered array of oligonucleotides that may be hybridized with fluorescently labeled material is inspected, using the microscope described in such patent. The individual specimens may be present in array densities for instance of 100 to 2000 or more specimens per square centimeter. An example would be biochips sold by Affymetrix, Inc., under the brand “GeneChip”, as is shown in FIGS. 12 and 13 of such patent. Such biochips may be pieces of borofloat glass fixed on black plastic cartridges permitting hybridization steps when connected to a fluidic station. In another case an ordered array of nucleic acid fragments is examined.

As is known to those skilled in the art, the DNA chips have specified DNA sequences arrayed in the chip material. The RNA transcripts of expressed genes will bind to complimentary gene sequences

Thereafter, and referring again to FIG. 3, in step 48 thereof one identifies those RNA transcripts that have bound to the chip array. These bound transcripts will fluoresce.

The extent of fluorescence of a bound transcript, if any, will vary with the degree to which the gene has been expressed; there will be no fluorescence with genes that are not expressed. The degree to which the gene has been expressed will, in turn, vary with the extent to which the expression was stimulated with electromagnetic radiation.

In step 50 of the process, the extent and intensity of fluorescence of the cell samples exposed to such electromagnetic radiation are compared with control cells that have not been so exposed. To the extent that, with any controlled system, electromagnetic radiation substantially increases the fluorescence, the promoter associated with such a sequence is a suitable promoter 22 for the process of this invention.

Thereafter, in step 52 of the process, gene maps are consulted to identify the particular gene and its associated promoter that is exhibiting the increased activity. As is well known to those skilled in the art, this promoter will be upstream of the nucleotide sequence in question.

Once the sequence of the promoter region that responds to such electromagnetic radiation is known, in step 54 of the process such promoter 22 can be synthesized and linked to any gene. Alternatively, or additionally, such promoter 22 can be isolated and cloned. The promoter 22 can also be “implanted,” i.e., inserted by homologous recombination.

Alternatively, in step 54, the aforementioned promoter 22 can be isolated if it is naturally occurring.

Alternatively, or additionally, in step 56 of the process chimeric or modified promoters 22 may be synthesized.

Regardless of how the promoter 22 is isolated and/or synthesized, such promoter that so responds to electromagnetic radiation is an “electromagnetic response element” (EMRE). These electromagnetic response elements then can be linked to any particular gene in step 58 of the process. Alternatively, the promoter can be linked to synthetic polynucleotide sequence that codes for the antisense transcript of a target gene.

In step 60 of the process, the construct produced in step 58, that contains the EMRE promoter so derived and linked to a desired gene, is then preferably transfected into any desired biological system. As is known to those skilled in the art, transfection is the introduction of foreign DNA into eukaryotic cells in tissue culture.

One may use prior art means for introducing foreign DNA into the eurkaryotic cells. In one embodiment, the process of U.S. Pat. No. 4,399,216, the entire disclosure of which is hereby incorporated by reference into this specification, is used. This patent describes and claims a process for inserting foreign DNA I into a suitable eucaryotic cell which comprises cotransforming said eucaryotic cell with said foreign DNA I and with unlinked foreign DNA II which codes for a selectable phenotype not expressed by said eucaryotic cell, said cotransformation being carried out under suitable conditions permitting survival or identification of eucaryotic cells which have acquired said selectable phenotype, said foreign DNA I being incorporated into the chromosomal DNA of said eucaryotic cell.

By way of further illustration and not limitation, one may use one or more of the processes disclosed in U.S. Pat. Nos. 4,6324,665, 5,149,636, 5,179,017, and the like. The entire disclosure of each of these United States patents is hereby incorporated by reference into this specification.

Referring again to FIG. 3, and in step 62 of the process depicted therein, different degrees and/or types of electromagnetic radiation are evaluated with the same cell, and/or with different cells, and/or with cells in different states. This process thus enables one to determine the biological activity of any electromagnetic signal in terms of genetic activity; and it provides a method for isolating the exact electromagnetic response elements that affect the genetic expression of the cell.

The signal used in the process of FIG. 3, in one embodiment, preferably is periodic, with time-varying fields. Thus, in this embodiment, the signal can be in the form of a sine wave.

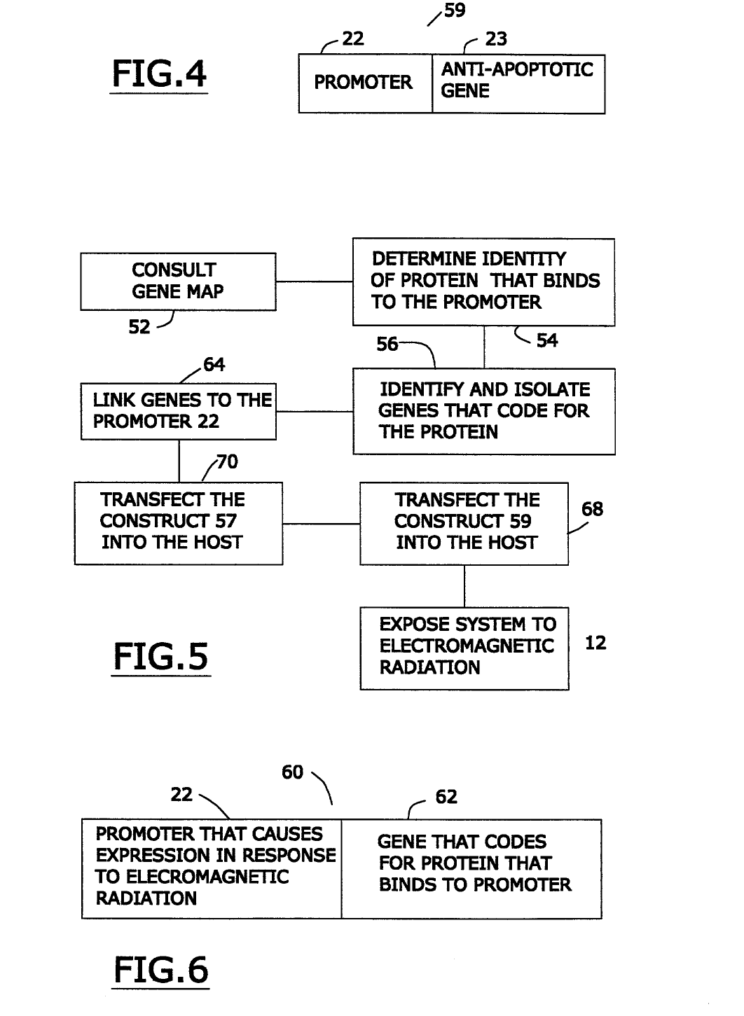

FIG. 4 illustrates a particular construct that can be made by the process of FIG. 3 and that extends the viability of cells containing such construct upon exposure to the appropriate electromagnetic field(s). Referring to FIG. 4, a promoter 22 that is responsive to electromagnetic radiation is linked to an anti-apoptosis gene 23 that prevents programmed cell death.

Anti-apoptosis genes are well known to those skilled in the art. Examples are the Bcl-2 gene, the Bcl-x gene, and the like. Reference may be had, e.g., to U.S. Pat. Nos. 6.284.880, 6,340,673, 6,172,047, 6,346,389, 6,245,523, 6,331,412, 6,077,709, 6,326,481, 6,280,966, 6,274,341, and the like. The entire disclosure of each of these United States patents is hereby incorporated by reference into this specification.

In one embodiment, the gene 23 may be the nucleotide sequence that codes for telomerease. As is known to those skilled in the art, telomerase is the enzyme that adds nucleotide sequences to chromosomes that are involved in programmed cell death, thereby inhibiting programmed cell death. As is disclosed in U.S. Pat. No. 5,565,638, the entire disclosure of which is hereby incorporated by reference into this specification, an understanding of the mechanisms by which normal cells reach the state of senescence, has begun to emerge. The DNA at the ends, or telomeres, of the chromosomes of eukaryotes usually consists of tandemly repeated simple sequences. Scientists know that telomeres have an important biological role in maintaining chromosome structure and function; and they have speculated that the cumulative loss of telomeric DNA over repeated cell divisions may act as a trigger of cellular senescence and aging, and that the regulation of telomerase, an enzyme involved in the maintenance of telomere length, may have important biological implications.

Telomerase is a ribonucleoprotein enzyme that synthesizes one strand of the telomeric DNA using as a template a sequence contained within the RNA component of the enzyme; see, e.g., Blackburn, 1992, Annu. Rev. Biochem., 61:113-129. Methods for detecting telomerase activity, as well as for identifying compounds that regulate or affect telomerase activity, together with methods for therapy and diagnosis of cellular senescence and immortalization by controlling telomere length and telomerase activity, have also been described. See, e.g., Feng, et al., 1995, Science, 269:1236-1241; Kim, et al., 1994, Science, 266:2011-2014; PCT patent publication No. 93/23572, published Nov. 25, 1993; U.S. patent application Ser. Nos. 08/330,123, filed Oct. 27, 1994; Ser. No. 08/272,102, filed Jul. 7, 1994; Ser. No. 08/255,774, filed Jun. 7, 1994; Ser. No. 08/315,214 and Ser. No. 08/315,216, both of which were filed Sep. 28, 1994; Ser. No. 08/151,477 and Ser. No. 08/153,051, both of which were filed Nov. 12, 1993; Ser. No. 08/060,952, filed May 13, 1993; and Ser. No. 08/038,766, filed Mar. 24, 1993. Each of the foregoing patent applications is referred to in U.S. Pat. No. 5,565,638.

Referring again to FIG. 4, and in another embodiment, the gene 23 may be a nucleotide sequence that promotes apoptosis, such as, e.g., an antisense sequence of the Bcl-2 gene. When expression of this antisense RNA is encouraged by the process of this invention, the antisense RNA so expressed will bind with the native Bcl-2 transcript, e.g., and prevent translation into functional protein. Apoptosis genes are also well known in the prior art and are described, e.g., in U.S. Pat. Nos. 5,888,764, 6,346,389, 6,077,709, 6,407,090, 6,395,510, 6,314,412, 6,326,481, 6,303,331, 6,284,880, 6,280,941, and the like. The entire disclosure of each of these is hereby incorporated by reference into this specification.

Referring again to FIG. 1, the transfected biological system with an ER element is exposed to electromagnetic radiation in step 26

More than one transfected DNA construct may be exposed to electromagnetic radiation in step 26. Thus, e.g. the host biological assembly may contain a second transfected biological construct that, upon being stimulated by electromagnetic radiation, expresses positive regulatory control factors that act upon the electromagnetic radiation promoter (EMRE).

Positive regulatory control factors are synthesized in the cell cytoplasm in response to stimulation by an electromagnetic field. These factors are expended from the cytoplasm as they translocate into the nucleus, bind to promoters, and effect the transcription of corresponding gene elements. Gene regulatory factors are well known and are described in, e.g., International patent publications WO0228873A2 and WO0155322A2, in U.S. Pat. Nos. 5,998,160 and 5,059,533, and in United States patent application US 2002/000979A1. The entire disclosure of each of these patent documents is hereby incorporated by reference into this specification.

The positive regulatory factor may be a positive transcription factor. Reference may be had, e.g. to U.S. Pat. Nos. 5,378,603 (LDL receptor gene), U.S. Pat. No. 5,256,545 (Sterol regulatory element), U.S. Pat. Nos. 5,215,910, 4,935,363, 5,558,999, 6,383,784, 6,369,038, 6,200,782, 6,051,376, 5,965,790, 5,891,631, 5,527,690, 5,498,696, and the like. The entire disclosure of each of these United States patents is hereby incorporated by reference into this specification.

Without wishing to be bound to any particular theory, applicants believe that, as positive regulatory control factors are removed from the cytoplasm, the rate of protein production decreases, thereby causing the cells to become refractory to further stimulation by electromagnetic radiation. It is an object of one aspect of this invention to continually replenish such positive regulatory control factors when the cells are stimulated by electromagnetic radiation.

This object can be accomplished by, e.g., transfecting into the nucleus a construct of the positive regulatory control factor bound to a promoter 22 that causes expression of the positive regulatory control factor upon exposure to electromagnetic radiation.

FIG. 3 illustrates how one can determine which promoters 22 react to electromagnetic radiation to substantially increase expression of a particular gene. FIG. 5 illustrates how to determine what positive regulatory control factors are associated with any particular promoter 22.

In the process depicted in FIG. 5, one may start with step 52 (see FIG. 3) in which one consults a gene map to determine the identity of a particular promoter 22 that has an extraordinary response to electromagnetic radiation. Once the identity of any particular desired promoter is known, one can determine by conventional means which proteins bind to such promoter; this determination can be made in step 54.

Thus, e.g., one may make such determination(s) in accordance with the process described in an article by Hana Lin et al, “Magnetic Field Activation of Protein—DNA Binding” (Journal of Cellular Biochemistry 70:297-303, 1998).

Once the identity of the positive transcription factors has been determined, then, in step 56, one can identify and isolate the genes that code for such proteins.

In step 58 of the process, the gene or genes that code for the proteins that bind to the promoter in question can then be linked to the electromagnetic response element promoter 22. The linked construct thus produced is illustrated in FIG. 6.

Referring to FIG. 6, it will be seen that the desired linked construct 60 is comprised of the desired promoter 22 linked to the desired gene 62. As will be apparent, when construct 60 is exposed to electromagnetic radiation, it will cause the expression of positive regulatory control factor binding proteins. The proteins thus expressed will replenish the proteins depleted by the stimulation of the first construct to express the first gene.

Without wishing to be bound to any particular theory, applicants believe that cells become refractory to electromagnetic radiation because, at least in part, the expression of the desired gene is dependent upon the supply of positive regulatory control factor, which is depleted by such expression of such desired gene. The living organism only replenishes this positive regulatory control factor as a relatively slow rate, as part of the cell cycle. By furnishing a supplementary source of such positive regulatory genetic control factor, the yield of the process is kept relatively constant and enhanced.

Thus, referring again to FIG. 5, in step 64 of the process the genes that code for the proteins in question are linked to the promoter 22. Thereafter, in step 68 of the process, the construct 60 is then transfected into the host.

One may introduce both of the promoter 22 constructs into the host by transfection means well known to those skilled in the art. Thus, e.g., reference may be had to U.S. Pat. No. 4,399,216 of Richard Axel that discloses construction of stably transformed eukaryotic cells transfected with foreign DNA nucleotide gene sequences. The products produced by the process of this patent, and other patents, include human recombinant insulin, erythropoeitin, growth hormones, clotting factors, viral antigens, cell surface antigens, antibodies, enzymes, and immune signaling factors, as some examples. Reference also may be had to U.S. Pat. Nos. 4,634,665, 5,149,636, and 5,179,017 of Richard Axel, the entire disclosure of each of which is hereby incorporated by reference into this specification.

Referring again to FIG. 5, in step 68 of the process, one may transfect the construct 59 into the host. The construct 59, which is illustrated in FIG. 4 and discussed elsewhere in this specification, is preferably a construct whose expression will inhibit programmed cellular death.

Referring again to FIG. 5, and in step 70 of the process, one may transfect the construct 57 into the host. The construct 57, one embodiment of which is illustrated in FIG. 2 in dotted line outline, expresses the desired gene 20.

In the process depicted in FIG. 5, three complementary phenomena will occur. In the first place, exposure to electromagnetic radiation will cause the expression of the desired gene 20. In the second place, exposure to such electromagnetic radiation will cause the production of positive regulatory factor protein(s), insuring an adequate amount of this rate-limiting material. In the third place, such exposure to electromagnetic radiation will also cause the production of the “anti-death” apoptosis proteins, thereby insuring the continued viability of the host protein pump.

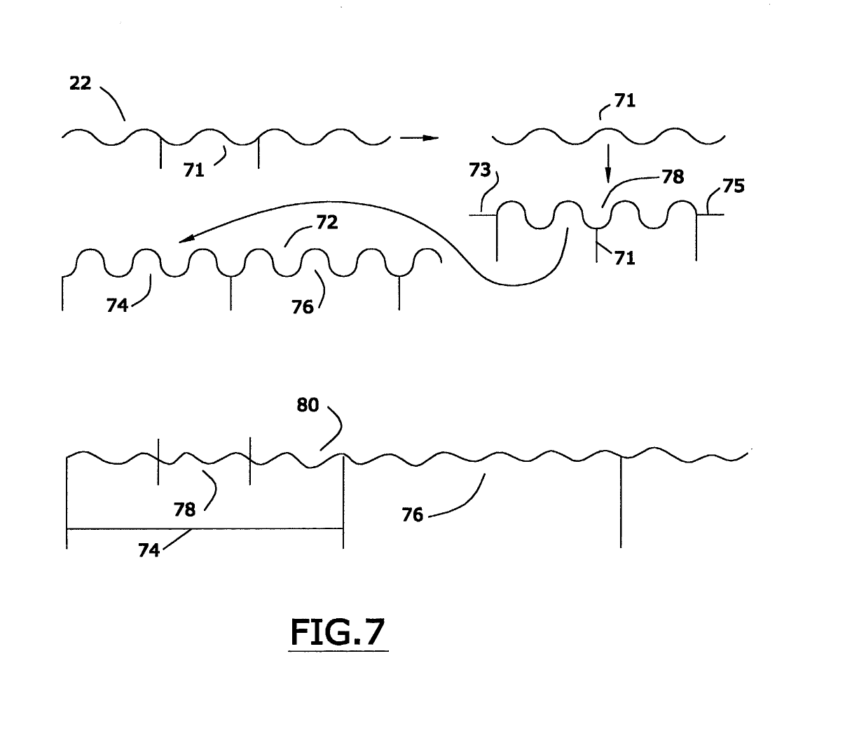

In another embodiment, illustrated in FIG. 7, transfection is used to target specific genes in a living organism with the active region(s) of the promoter 22.

Referring to FIG. 7, it will be seen that promoter 22 is comprised of a multiplicity of polynucleotide DNA sequences. Each promoter 22 is comprised of an active binding region 71 to which the positive regulatory proteins (not shown) bind.

By conventional means, such as restriction enzymes, the binding region 71 for any particular promoter 22 of interest is excised. One can thereafter target a particular gene in a living biological system.

The particular gene assembly is identified as assembly 72. As will be apparent, assembly 72 is comprised of a promoter 74 and a targeted gene 76.

The binding region 71 can then be linked with sticky ends 73 and 75. The sticky end construct so produced, construct 78, can then be used to combine with promoter 74.

This process can be effected by homologous recombination, a technique well known to those skilled in the art; via this process, one can produce substantially any desired protein from an in situ gene. Furthermore, any cell so transformed may be reintroduced into a host oranism from which the transformed cell was derived in order to produce the desired gene product in response to one or more selected electromagnetic fields.

Homologous recombination is genetic exchange between DNA molecules that have identical or nearly identical base sequences; and it also often referred to as general recombination. Reference may be had, e.g., to U.S. Pat. Nos. 6,348,327, 6,255,113 (homologous sequence targeting), U.S. Pat. No. 6,074,853 (sequence alternation using homologous recombination), U.S. Pat. No. 5,948,653 (sequence alternation using homologous recombination), U.S. Pat. No. 5,763,240 (in vivo homologous sequence targeting), and the like. The entire disclosure of each of these United States patents is hereby incorporated by reference into this specification.

By way of further illustration, the process can be effected by the techniques patented by Transkaryotic Therapies, Inc. in their United States patents, some of which are discussed below. These patents include U.S. Pat. Nos. 6,355,241, 6,214,622, 6242,218, 6,187,305, 6,063,630, 6,083,725, 6,054,288, and the like. The entire disclosure of each of these United States patents is hereby incorporated by reference into this specification.

Thus, e.g., U.S. Pat. No. 6,063,630 describes and claims a method of producing a clonal cell strain of homologously recombinant secondary somatic non-immortalized cells of vertebrate origin selected from the group consisting of mammalian secondary fibroblasts, keratinocytes, epithelial cells, endothelial cells, glial cells, neural cells, lymphocytes, bone marrow cells, muscle cells, hepatocytes and precursors thereof. This method comprises the steps of: a) transfecting a primary or secondary somatic non-imnmortalized cell of vertebrate origin with a DNA construct comprising exogenous DNA which includes a DNA sequence homologous to a genomic DNA sequence of the primary or secondary cell, thereby producing a transfected primary or secondary cell, and wherein said exogenous DNA further comprises a DNA sequence that encodes a therapeutic product selected from the group consisting of enzymes, cytokines, hormones, antigens, antibodies, clotting factors, regulatory proteins, transcription proteins, and receptors; b) maintaining the transfected primary or secondary cell under conditions appropriate for homologous recombination between a DNA sequence in the DNA construct and genomic DNA to occur, thereby producing a homologously recombinant primary or secondary cell; and (c) culturing the homologously recombinant primary or secondary cell under conditions appropriate for propagating the homologously recombinant primary or secondary cell, thereby producing a clonal cell strain of homologously recombinant secondary somatic non-imnmortalized cells, wherein the clonal cell strain supplies said therapeutic product. The entire disclosure of this United States patent is hereby incorporated by reference into this specification.

Thus, e.g., U.S. Pat. No. 6,187,305 describes and claims a method of providing a therapeutic product in an effective amount to a mammal comprising the steps of: a) providing a DNA construct comprising: 1) exogenous DNA encoding a product to be expressed in primary or secondary cells of mammalian origin; 2) DNA sequences homologous with genomic DNA sequences in the primary or secondary cells of mammalian origin; and 3) DNA sequences encoding at least one selectable marker; b) transfecting primary or secondary cells obtained from a mammal with the DNA construct provided in (a), thereby producing primary or secondary cells containing the DNA construct provided in (a); c) maintaining primary or secondary cells produced in (b) under conditions appropriate for homologous recombination to occur between DNA sequences homologous with genomic DNA sequences and genomic DNA sequences, thereby producing homologously recombinant primary or secondary cells of mammalian origin having the DNA construct of (a) integrated into genomic DNA of the primary or secondary cells; and d) introducing said homologously recombinant primary or secondary cells produced in (c) into a mammal in sufficient number to produce an effective amount of the therapeutic product in the mammal, wherein said recombinant primary or secondary cells are from the same species as said mammal The entire disclosure of this United States patent is hereby incorporated by reference into this specification.

Thus, e.g., U.S. Pat. No. 6,214,622 describes and claims a method of altering expression of a gene that is present in a non-immortalized primary or secondary cell, said method comprising the steps of: (a) introducing into said primary or secondary cell a DNA construct comprising a regulatory region under conditions suitable for homologous recombination, whereby said regulatory region is inserted into or replaces all or a portion of the regulatory region of the gene and alters expression of said gene, and (b) culturing said primary or secondary cell to produce a clonal cell strain of homologously recombinant non-immortalized secondary cells in which expression of said gene is altered, wherein the clonal cell strain supplies the product of the gene that is expressed. The entire disclosure of this United States patent is hereby incorporated by reference into this specification.

Referring again to FIG. 7, the product thus produced is identified as construct 80, which now includes a modified promoter 74 that contains the active region 70 that is especially responsive to electromagnetic radiation. Thus, by the process depicted in FIG. 7, one may make naturally occurring genes in a biological organism especially responsive to electromagnetic radiation.

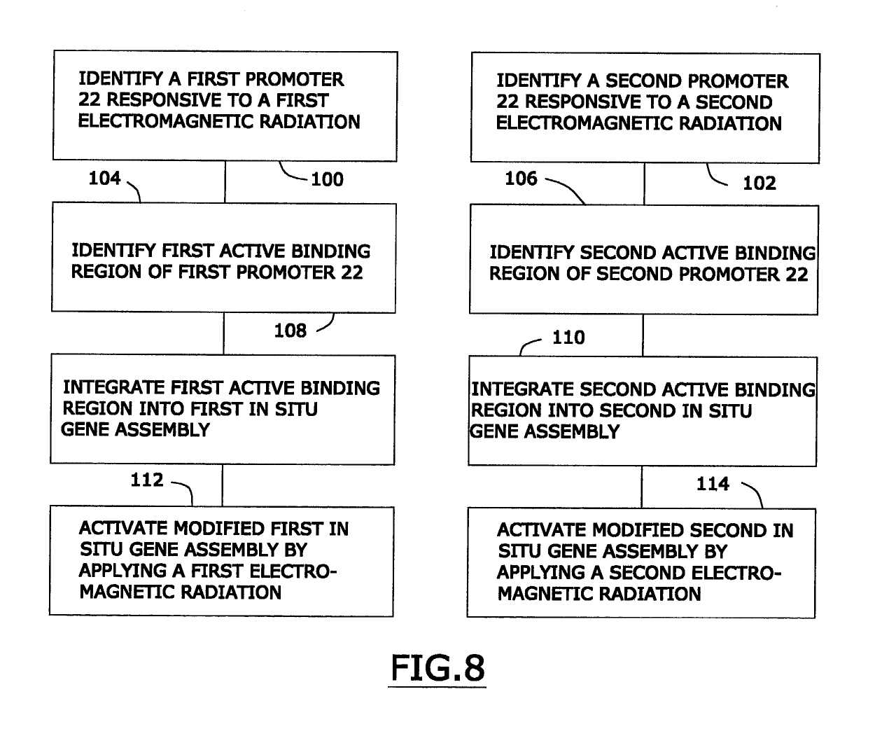

One may create a living organism with a multiplicity of naturally occurring gene assemblies responsive to different electromagnetic radiations. This process is depicted in FIG. 8.

May different types of biological organisms may so be created with such naturally occurring gene assemblies responsive to different electromagnetic radiations. Some of these biological organisms/systems are described below.

The biological system created by the process of this invention may also comprise an implantable position and motion sensor. See, e.g., U.S. Pat. No. 4,846,195, the entire disclosure of which is hereby incorporated by reference into this specification.

The biological system of this invention may comprise an implantable microstimulator housed within a hermetically sealed housing. See, e.g., U.S. Pat. No. 5,358,514, the entire disclosure of which is hereby incorporated by reference into this specification.

The biological system may also comprise an implantable drug infusion pump. See, e.g., U.S. Pat. No. 5,586,629, the entire disclosure of which is hereby incorporated by reference into this specification.

The biological system of this invention may also comprise an implantable access device which permits the introduction of an external filament (such as a catheter, guide wire, or optical fiber) into the system; see, e.g., U.S. Pat. No. 5,607,393, the entire disclosure of which is hereby incorporated by reference into this specification.

The biological system created by the process of this invention may also comprise a surgically implanted reciprocating pump. See, e.g., U.S. Pat. No. 5,675,651, the entire disclosure of which is hereby incorporated by reference into this specification. Reference also may be had to U.S. Pat. No. 5,675,162.

The biological system of this invention may also comprise an implantable in-line pressure check valve. See, e.g., U.S. Pat. No. 5,725,017, the entire disclosure of which is hereby incorporated by reference into this specification.

The biological system of this invention may comprise a leadless, multisite implantable stimulus and diagnostic system, such as the system disclosed in U.S. Pat. No. 5,814,089; the entire disclosure of such patent is hereby incorporated by reference into this specification. This patent discloses an implantable stimulating system comprising an implantable stimulating unit and an implantable controller.

The biological system of this invention may also comprise an implantable, refillable, rate-controlled drug delivery device comprised of a hollow reservoir and a drug delivery tube. See, e.g., U.S. Pat. No. 5,836,935, the entire disclosure of which is hereby incorporated by reference into this specification. Reference also may be had, e.g., to U.S. Pat. Nos. 5,607,418, 5,976,109, 6,048,328, the entire disclosure of each of which is hereby incorporated by reference into this specification.

The biological system of this invention may also contain an monitoring arrangement for monitoring and controlling a device implantable in a human body. Reference may be had, e.g., to U.S. Pat. No. 5,879,375, the entire disclosure of which is hereby incorporated by references into this specification.

The biological system of this invention may also contain an implantable ultrfiltration device. See, e.g., U.S. Pat. No. 5,902,336, the entire disclosure of which is hereby incorporated by reference in to this specification.

The biological system created by the process of this invention may also contain a low power current to frequency converter for use in implantable sensors. See, e.g., U.S. Pat. No. 5,917,346, the entire disclosure of which is hereby incorporated by reference into this specification.

The biological organism created by the process of this invention may also comprise an implantable sensor such as, e.g., an implantable device for sensing in vivo the level of at least one blood constituent in mammalian tissue. See, e.g., U.S. Pat. No. 5,995,860, the entire disclosure of which is hereby incorporated by reference into this specification.

The biological system of this invention may comprise daisy chainable sensors and stimulators that are implantable in living tissue. See, e.g., U.S. Pat. No. 5,999,848, the entire disclosure of which is hereby incorporated by reference into this specification.

The biological system of this invention may comprise a low power rectifier circuit for an implantable medical device. See, e.g., U.S. Pat. No. 5,9999,849, the entire disclosure of which is hereby incorporated by reference into this specification.

The biological system of this invention may comprise implantable electrodes. See, e.g., U.S. Pat. No. 6,038,480, the entire disclosure of which is hereby incorporated by reference into this specification.

The biological system of this invention may comprise an implantable adaptive brain stimulation system comprised of an implantable brain stimulator. See, e.g., U.S. Pat. No. 6,066,163, the entire disclosure of which is hereby incorporated by reference into this specification.

Referring again to FIG. 8, and in step 100 thereof, a first promoter 22 is identified that is responsive to a first electromagnetic radiation.

In step 102, a second promoter 22 is identified that is responsive to a second electromagnetic radiation.

In step 104, the active binding region of the first promoter 22 is identified. In step 106, the active binding region of the second promoter 22 is identified.

In step 108, the first active binding region of the first promoter is integrated into a first specified gene region, as disclosed in FIG. 7. In sep 110, the second active binding region of the second promoter is integrated into a second specified gene region.

Alternatively, or additionally, one may integrate gene regions from third, fourth, and fifth promoters (not shown) into specified in situ gene assemblies; each of these integrated gene assembly constructs will then be optimally responsive to a specified electromagnetic radiation.

Referring again to FIG. 8, in step 112, one may activate the modified first in situ gene assembly by applying a first electromagnetic radiation to which such gene assembly is optimally responsive. Thereafter, in 114, one may activate the modified second in situ gene assembly by applying a second electromagnetic radiation to which such gene assembly is optimally responsive.

As will be apparent, a biological organism with a multiplicity of such integrated gene assemblies in situ can be “tuned” by selectively applying one or more electromagnetic radiations at one more points in time to elecit different responses and, in particular, to eleicit desired differential gene regulation and selection of entire genetic programs.

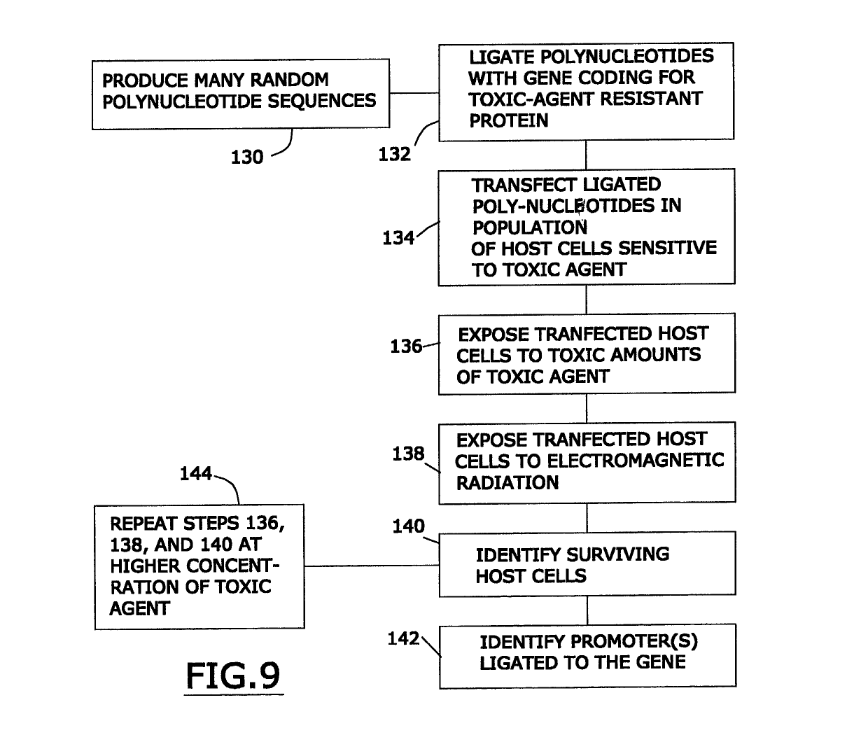

FIG. 3 illustrates a process for identifying naturally occurring promoters 22 that increase gene expression in the presence of electromagnetic radiation. FIG. 9 illustrates a process for identifying synthetic promoters 22 that will also increase gene expression in substantially the same manner.

Referring to FIG. 9, and in step 130 thereof, a multiplicity of random polynucleotide sequences of varying lengths are produced. It is preferred that these sequences be at least 4 base pairs long.

In step 132, the random polynucleotides produced in step 130 are integrated into a promoter (see FIG. 7) and then ligated with a gene that codes for a protein that confers resistance to a specified toxic agent.

In step 134, the soup of promoters containing the random polynucleotide sequences of step 130 are ligated to the gene that codes for resistance to a toxic agent. The constructs so produced are transfected into a population of host cells that are normally killed by a specified toxic agent. Thus, e.g., the host cells may be prokaryotic cells, eukaryotic cells, parts of whole organisms, plants, animals, etc.

In step 136, the transfected host cells are exposed to the toxic agent at concentrations that would normally kill host cells or inhibit their growth. Thereafter, in step 138, the transfected biological system is exposed to electromagnetic radiation.

Only certain of the transfected host cells, i.e., those transfected host cells that produce protein that deactivates the specified toxic agent, will survive. In step 140, the surviving viable transfected host cells are identified.

From the surviving entities, the gene/promoter constructs are recovered and the promoter elements are sequenced and compared with the original, native promoter. Thus, those sequences that differ from the native promoter are the sequences that are electromagnetically responsive. This identification process occurs in step 142.

In step 144, one may take the soup of surviving host cells and repeat steps 136, 138, and 140 at successively higher concentrations until fewer and fewer of such clones survive. The last such clone to survive will contain the synthetic promoter that is most effective in upregulating a gene ligated to it

With a similar process, under the same biological conditions, one may repeat the process with different electromagnetic signals to identify the entire universe of signal specific electromagnetic response elements.

With the use of the processes depicted in FIGS. 1-9, one can identify a series of promoters 22 that afford varying degrees of upregulation in response to either the same electromagnetic field or different electromagnetic fields. For any particular electromagnetic signal, one can identify the promoter most responsive to that signal. Correspondingly, for any particular promoter, one can identify the electromagnetic field most responsive to that promoter.

Thus, e.g., by way of illustration, one may identify the promoters from the DNA chip array that are upregulated in response to a particular signal and, thereafter, and identify that promoter within this group that has the highest degree of activity when compared with the control; this will allow one to identify the most response electromagnetic response element within such optimum promoter, for that particular signal.

Thus, by way of further illustration, for any particular electromagnetic response element (such as, e.g., the optimum electromagnetic response element), one can link this EMRE to a gene that makes a detectable product.

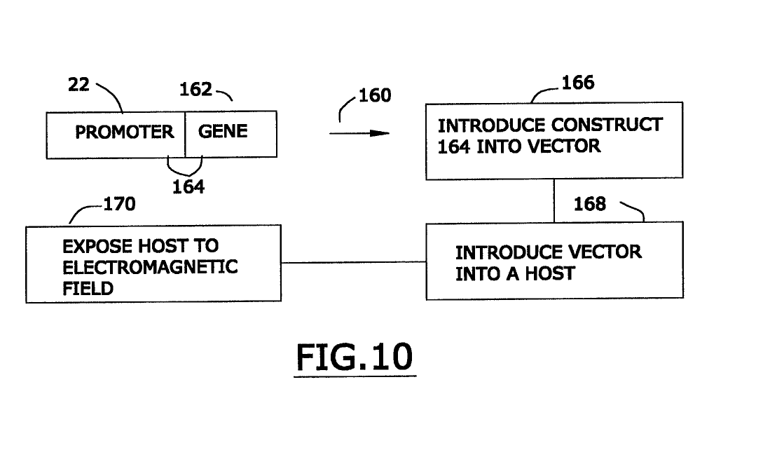

FIG. 10 illustrates one preferred process 160 for producing a desired protein that can be used ex vivo. In step 162 of this process, a desired gene is ligated to a promoter 22 that has been found to be especially active in expressing such gene when contacted with electromagnetic radiation. For purposes of reference, the gene so ligated is identified as construct 164

In step 166 of the process, the construct 164 is introduced into a vector. Thereafter, in step 168, the vector is introduced into a host.

In step 170 of the process, the host is exposed to an electromagnetic field and optimized, in accordance with the process of this invention, to maximize expression of the gene.

The following discussion relates to the improvements described in FIGS. 1-10.

As the genomes of humans and other organisms continue to be elucidated, many more useful products, as yet unknown, are becoming available; these products often are produced by recombinant gene technology, including stable transfection with foreign genes, and the homologous recombination techniques described elsewhere in this specification.

Many of these new products are powerful selective modifiers of cellular physiology that are currently produced by cells in only scant amounts.

It is one object of this invention to provide electromagnetic methods to regulate the expression of endogeneous cellular nuclear transcription factors for the purpose of regulating the cellular production of polypeptide chains ligated to gene promoter regions that are electromagnetic response elements. It is another object of the invention to provide a general method for the improved production of any desired recombinant gene product obtained from an organism, with specific reference to in-vitro cell culture systems.

In one embodiment, the present invention relates to the production of recombinant DNA gene products by eukaryotic cells stably transformed with foreign DNA, coding for the selected amino acid sequences. In particular, the present invention, in one embodiment, provides a method to increase the production of desired products by cells that have been transformed with foreign DNA, wherein the cells contain gene promoter regions that are responsive to a remotely applied time-varying electromagnetic field such that the production of gene product is upregulated.

In one embodiment, the present invention provides a method to regulate the activity of genes from an external electromagnetic source. In particular, the present invention uses biologically active electromagnetic fields that act as non-physical biological response modifiers

In one embodiment, the present invention provides a distinct genetic method to regulate the transcription of selected nucleotide sequences using a time-varying electromagnetic field. The method substantially relies on the tendency of a biological system to adapt to changes in ambient electromagnetic conditions. Changes in genetic programming mediate adaptation. De novo production of nuclear transcription factors and binding thereof to specific gene promoter regions mediate genetic reprogramming. Gene promoter genes that bind electromagnetic responsive nuclear transcription factors are termed electromagnetic response elements. Desired gene nucleotide sequences are ligated downstream to electromagnetic response elements. DNA elements so constructed are transfected into host biological systems. Application of and adaptation to a biologically active electromagnetic field elicits a transient increase in the production of the desired gene product above steady-state conditions by a biological system so engineered.

The system of electromagnetic field and genetically engineered biological responses comprises a method and process for the construction of a recombinant DNA protein production pump. Novel application of biologically active electromagnetic fields increases the efficiency of the pump toward a continually sustained elevation of steady-state protein production beyond current yields.

In one embodiment, a specific promoter region of the c-myc structural gene is ligated upstream to a desired foreign nucleotide sequence coding for a polypeptide chain. The resulting recombinant DNA structure is then stably integrated into the genome of a host cell. Exposure of the cultured host cell to a weak nonthermal time-varying electromagnetic field triggers a series of intracellular events that produce nuclear transcription factors that bind to the c-myc promoter and prompt the transcription of the ligated nucleotide sequence. Foreign genes that are stably integrated into the genome of a host cell that are otherwise subject to negative regulatory controls are produced in very limited amounts. The present invention overcomes this limiting genetic control, thereby increasing the production of desired polypeptide sequences.

In another embodiment, a specific portion of the promoter element of the HSP70 structural gene is ligated upstream to a desired foreign nucleotide sequence coding for a polypeptide chain. The resulting recombinant DNA structure is then stably integrated into the genome of a host cell. Exposure of the cultured host cell to a high-frequency thermal time-vary electromagnetic field triggers the production of heat shock factors that bind to the HSP70 promoter and prompt the transcription of the ligated nucleotide sequence. Alternatively, through transactivation by the c-myc gene product contained by a host cell, the HSP70 promoter gene additionally prompts transcription of a desired nucleotide sequence by cultured cells exposed to a weak nonthermal time-vary electromagnetic field.

In one embodiment of this invention, there is provided a process for the dual activation of transcription of a desired nucleotide sequence ligated to the promoter region of HSP70 by exposure of a cultured host cell to either a weak nonthermal electromagnetic field or a high-frequency thermal electromagnetic field. This embodiment allows for the increased production of desired polypeptide sequences by two distinct mechanisms in the same host cell to overcome inherent genetic control that limits transcription of desired DNA nucleotide sequences.

A major limitation of any culture system for the production of desired polypeptide sequences is the viability of the culture itself. In another embodiment of the invention, specific portions of either the c-myc promoter element or the HSP70 element are ligated upstream to a gene or genes that code for a protein that prevent programmed cell death. These genes are termed anti-apoptosis genes. In this embodiment of the invention recombinant DNA gene constructs are stably integrated into a host cell. Exposure of the host cell in culture to a weak time-varying electromagnetic field increases the production of anti-apoptosis proteins and delays the programmed cell death of the cultured host cells in particular, and extends the productivity of the host culture system in general.

In another embodiment, integration of DNA constructs into a host cell genome consisting of electromagnetic response elements ligated to genes that code for genetic regulatory proteins provides a method of regulating the transcription of selected genes. Response elements ligated to positive control elements upregulate gene expression when the host cell is exposed to a time-varying electromagnetic field. Similarly, response elements ligated to negative control elements downregulate gene expression when the host cell is exposed to a time-varying electromagnetic field. Downregulation is accomplished in another embodiment by ligating a response element to a synthetic nucleotide sequence that is the antisense sequence of a selected gene. The transcription of the antisense sequence binds to the transcript of the selected gene and prevents translation into a polypeptide sequence, thereby effecting downregulation of the gene when the host cell is exposed to a time-varying electromagnetic field.

In one embodimnt, transcription of selected genes is controlled indirectly by nucleotide sequences that code for regulatory proteins that bind to the promoter regions of other genes. This is referred to as genetic transactivation. See, e.g., U.S. Pat. Nos. 5,945,578, 5,482,852, and 5,222,341, the entire disclosure of each of which is hereby incorporated by reference into this specification.

In another embodiment, downregulation of genetic activity of a selected gene is achieved by a trans-acting anti-sense nucleotide sequence. In another embodiment, cells are transformed with multiple electromagnetic response elements, each of which is responsive to a specific time-varying field with differing electromagnetic parameters.

In yet another embodiment, positive and negative control of gene transcription is simultaneously effected in the same cell.

In yet another embodiment, a process is provided for producing recombinant DNA constructs that act as vehicles for the genetic transformation of a host cell. In this embodiment, a response element is ligated to any desired gene, thereby providing a generalized mechanism to upregulate the transcription of selected nucleotide sequences.

In yet another process, recombinant DNA gene products are produced and collected from host cell cultures. In general, the process involves constructing a recombinant DNA vehicle for stable transformation of host cells. Specialized promoter regions in the DNA vehicle may be employed to enhance the transcription of the transforming nucleic acid sequence. Once transformed, host cells are cultured to produce desired products. Specialized methods may be used to promote the viability and productivity of the host cell culture system. Product is then collected from the culture and purified for use.

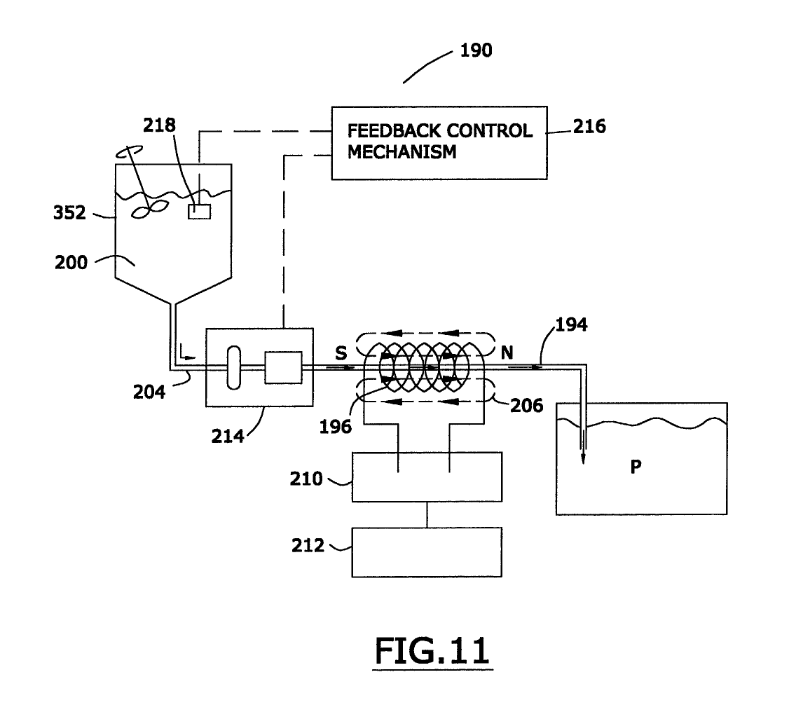

FIG. 11 is a schematic diagram of a self-regulating reactor vessel 190 for the optimum production of a desired gene product. The reactor vessel 352 includes a collecting conduit 194 that has attached means for producing an electromagnetic field 196 and means 218 for monitoring the stimulatory effect of the electromagnetic field on gene production. A reservoir 200 of culture medium and host cells. is adapted to flow the cell culture through a conduit 204 at a selected flow rate as a final process before culture harvesting. The conduit 204 is surrounded by electromagnetic coil means 196 that provide a uniform magnetic field 206 by which the cultured host cells are treated. The coil is energized by a power source that provides current to the coil. The coil 196 generates a weak athermal magnetic field in one embodiment. In another embodiment, not shown, the coil can be modified or superimposed with another coil that produces a high-frequency thermal field capable of rapidly heating the culture medium. In another embodiment, the superimposed coil generates a substantially static magnetic field. Alternatively, a permanent magnetic field source is provided.

Referring again to FIG. 11, a power source 210 and circuit means 212 (such as a function generator) for producing selectable electromagnetic field parameters are provided.

A flow rate controller 214 controls the rate at which culture medium containing host cells passes through the treatment conduit enroute to culture harvesting. A feedback control mechanism 216 is provided that monitors the stimulatory effect of the magnetic field and regulates the flow rate of the culture through the conduit and the magnetic field parameters in the conduit so that production of the desired gene product is optimized at a constant rate.

Many types of physiological responses have been seen at all levels of biological organization following exposure to electromagnetic fields. At the cellular level it is widely held that real-time energetic interactions of the field with cell membrane components trigger responses that alter intra-cellular cytoplasmic metabolism. These changes in turn produce responses within the nucleus of the cell that ultimately affect genetic expression.

Many electromagnetic field parameters produce measurable biological responses at all levels of biological organization, ranging from molecular assemblages involved in electron transport to observable responses in whole organisms including humans. These field parameters include, but are not limited to signals that have a continuous wave form output; a discontinuous intermittent pulsed wave form output; a pure sine wave frequency output; a high-frequency carrier wave form modulated by a low-frequency sine wave element; a complex wave form output composed of square and/or triangle wave form elements; unipolar/bipolar pulses; an electromagnetic field that is nonpropagating; an electromagnetic field frequency that is propagating; a complex wave form comprising at least one static magnetic field element in combination with at least one time-varying magnetic field element; a circularly polarized electromagnetic field wave form; a linearly polarized electromagnetic field wave form, and the like. In addition, static magnetic fields, static electric fields, and pure time-varying electric fields produce biological responses. Electromagnetic signals that produce biological effects are also broadly classified into thermal fields that produce heat-mediated responses, and athermal fields that produce responses without the production of biologically significant levels of heat.

Athermal fields that produce observable biological responses are generally termed “biologically active” fields. Such fields act as biological response modifiers. Biological response modifiers are often small molecules that interact with naturally occurring endogenous physiological processes or biological structures. In the present invention, the biological response modifier is preferably an exogenous non-molecular agent, an externally applied electromagnetic field. Additionally, biologically active fields evoke adaptive physiological responses in responsive systems to which they are applied

A general characteristic of a biologically active athermal field is that the energy of the field deposited into an exposed biological system is preferably orders of magnitude below the thermal noise, kT, inherent to the cell at biologically functional temperatures, where k is the Boltzman constant and T is the absolute temperature in Kelvin. The mechanism by which a biological system can discern such a weak signal amidst thousands of times more electromagnetic noise, or static, due to thermal Brownian movement of charged particles in the cell is the central mechanistic problem in bioelectromagnetics. While no electromagnetic parameters have been shown to be absolutely necessary for biological activity, there is virtually no end to the number and combination of signal parameters that are sufficient for biological activity. Nonetheless, it is generally accepted that certain ranges of frequencies and amplitudes of an electromagnetic field are particularly biologically active. These ranges are termed frequency windows and amplitude windows. For a time-varying nonpropagating electromagnetic field, these windows exist at amplitudes approximately below 3 milliTesla (20 to 30 gauss) and at frequencies approximately below 300 cycles/second. Biological effects of time-varying electromagnetic fields can also be masked or neutralized by fields that are aperiodic and composed substantially of electromagnetic white noise, which is characterized by a substantially uniform distribution of energy across a wide frequency band.

A limiting feature of any athermal biologically active electromagnetic field is lack of specificity for a desired biological effect. There is no known method provided by the prior art to select particular signal parameters to produce selective biological responses, particularly at the level of genetic expression. Rather, once a membrane-mediated response is triggered by an electromagnetic field, the response observed in the biological system is largely determined by the state of the organism at the time of exposure.

The present invention positions specific nucleic acid sequences in the genome that are responsive to electromagnetic fields such that selected states of genetic expression are obtained. In effect, the nonspecific membrane-mediated intracellular events triggered by a biologically active field that result in the expression of nuclear transcription factors are transduced by an electromagnetic response element situated upstream of a desired nucleotide sequence targeted for regulation from an external electromagnetic source. The present invention introduces a mechanism for attaining biological specificity from an otherwise nonspecific biologically active electromagnetic field. The invented method of specificity is, therefore, embodied in the position of an electromagnetic response element relative to a desired gene sequence. Alternatively, and in addition to positional harnessing of nuclear transcription factor activation, specificity from a biologically active field can be attained by direct interaction of the field with specific nucleotide sequences within the electromagnetic response element itself.

Regulation of genetic expression from an external electromagnetic source has generalized application in biotechnology. Processes in biotechnology involving both prokaryotic and eukaryotic organisms are equally applicable. The present invention discloses a general method for electromagnetic regulation of gene activity that can be applied, e.g., to the biofermentation industry as a whole, and the production of recombinant DNA gene products in particular. In addition, as the elucidation of the genomes of organisms including humans continues, the present invention can be applied to virtually any gene product for which the importance and utility of electromagnetic regulation from an external source is evident. This applies equally to whole organisms such as transgenic plants and transgenic animals as well, in addition to fractionated biological systems thereof.

Referring to FIG. 12, a transactivating electromagnetic response element promotes the production of a cis, or colinear gene product that regulates the production of another noncolinear gene, or trans gene in a differing region of the genome. The basic method for transregulation, or cross-regulation of gene products using electromagnetic response elements and an externally applied electromagnetic field is illustrated in FIG. 12 In step 300 of the process depicted in FIG. 12, a promoter 22 is ligated to polynuceotide antisense sequence that transdeactivates a another targeted gene. This occurs by the mechanism of transactivation, which is well known t those skilled in the art. In transactivation, one gene product regulates another gene product that is non-colinear with it.

In step 302, an electromagnetic field adapted to maximize the effect of promoter 22 is applied to the construct 303 thereby, in step 304, transregulating gene 306. This transregulation produces an antisense sequence that binds to sense sequence of gene 306 and thus prevents the translation thereof into a functional protein. Put another way, the electromagnetic response element under the action of a magnetic field promotes the production of a gene product that regulates the transcription of another gene through the binding of the electromagnetically responsive gene product to the promoter region of the trans gene. The electromagnetically responsive protein is ether a positive or negative control element. In addition, the promoter of the trans gene may itself be an electromagnetic response element, which would provide a means for amplifying the effect of trans-regulation by applying an electromagnetic field. Cross regulation of the trans genes by the synthesis of positive or negative control proteins offers a means of providing fine-tuned, self-regulating feedback controls between and among genes.

In another embodiment, not shown, the gene 304, instead of producing an antisense transcript, produces a protein that has a negative regulatory effect upon the gene 306; in one embodiment, the protein binds to the promoter 308 and down regulates expression of gene 306.

In yet another embodiment, the gene 301 may produce a protein that is a positive regulatory factor that upregulates the expression of gene 306.

The production of selected polypeptides can be increased in cell culture by inserting electromagnetic response sequences into promoter regions otherwise lacking them, ligating a selected nucleotide sequence downstream to an electromagnetic response element so constructed, transfecting a host cell with the recombinant DNA agent so constructed, and exposing the cell culture to an electromagnetic field.

The present invention employs magnetic fields, although it is understood that pure electric fields may also be applicable. Biolocally active fields include fields that are propagating/nonpropagating; static/time-varying; pure frequency/complex frequency; single field systems/multi-field systems; continuous output/intermittant output; thermal/nonthermal; linearly polarized or circularly or elliptically polarized.

The simplest pure frequency electromagnetic field is the sine wave. So-called extremely low frequency fields have a frequency content of less than 300 Hz. Such fields are delivered to biological systems as a continuous output or in intermittant pulses. Fields can also be delivered via an amplitude modulated high-frequency carrier wave. Complex frequency fields have distinct shapes characterized by distinct rise times and fall times. The basic examples of a complex frequency wave form are the pure square wave and the pure triangle wave. By Fourier series analysis, a pure square wave contains an infinite series of sine waves of decreasing amplitudes at odd harmonics. In like fashion, a pure triangle wave contains an infinite series of sine waves of decreasing amplitudes, but at all even harmonic frequencies. Combinations of square and triangle waves produce complex shapes such as trapezoids, which contain both even and odd harmonic components. Sine waves are described by the parameters of frequency and amplitude. Square waves and triangle waves are described by rise times and fall times, amplitude, and pulse repetition rate. Additionally, square waves have a plateau phase characterizing pulse width duration. Amplitude-modulated carrier waves are characterized by the frequency and amplitude of the modulation and the frequency of the carrier. In addition, if the field is delivered in bursts or envelops, a pulse repetition rate is described. Multi-field magnetic delivery systems generally consist of a static magnetic field in combination with a time-varying magnetic field. The fields may be either perpendicular or parallel to one another. For parallel field systems, the so-called cyclotron resonance frequency for a selected ion can be defined Such field systems can be tuned to selected ions to produce biologically active fields. For perpendicular field systems, an ion precession frequency can be similarly defined for selected ions to construct biologically active fields.

The description set forth in the first portion of this specification relates primarily to the effect of electromagnetic fields upon eukaryotic cells. As is known to those skilled in the art, eukaryotes have a true nucleus surrounded by a nuclear membrane and containing the genetic material within multiple chromosomes. However, the process of the invention also is applicable to prokaryotes, such as bacteria, which are simple unicellular organisms that lack a discrete nucleus surrounded by a neuclar membrane. Prokaryotes generally contain their genetic material within a single chromosome.

By way of illustration and not limitation, magnetic fields enhance the expression of messenger RNA of sigma 32 in E. coli; see, e.g., an article by P. Cairo et al entitled “Magnetic field exposure enhances mRNA expression . . . ,” J Cell Biochem 1998 Jan 1; 68(1), pages 1-7. The sigma 32 protein interacts with RNA polymerase to help it recognize a variety of stress promoters in the cell. As is disclosed in the abstract of this article, “ . . . the cells respond to MFs in a manner similar environmental stressors such as heat.” It appears that this phenomenon is substantially similar to the expression of heat shock protein in eukaryotic cells and its enhancement by electromagnetic fields.

By way of further illustration, a sinusoidal magnetic field alters protein synthesis in E. coli; in particular, exposure to such fields elevates the level of the alpha subunit of RNA polymerase. See, e.g., an article by E. M. Goodman et al., “Altered protein synthesis in a cell-free system exposed to sinusoidal magnetic field,” Biochim Biophys Acta 1993 Sep 3, 1202, pages 107-112. In the experiment described in this article, a cell-free transcription/translation system derived from Eschrichia coli was exposed to a 72 Hertz sinusoidal field in the range of from 0.07 to about 1.1 milliTesla (rms) for a period of from about 5 to about 60 minutes. The magnetic fields used in this experiment were about as strong as the relatively weak magnetic field of the earth. When a exposure time of 10 minutes was used, demonstrable effects were demonstrated with a magnetic field of only 0.1 milliTesla. As was disclosed in this article, “Weaker fields must be applied longer to produce an effect.” The experiment of this article clearly demonstrates that “ . . . an intact membrane is not an absolute requirement for transducing magnetic bio-effects.”

A reactor 1000 adapted to utilize the process of this invention to treat prokaryotic cells is illustrated in FIG. 13. By the use of the processes described in the first part of this specification, one may identify the electromagnetic response elements that naturally occur in prokaryotic cells and, for such elements, optimize the electromagnetic signal(s) that activate such elements. Similarly, by the processes described earlier in this specification, one may also determine which synthetic nucleotide sequences act as electromagnetic response elements for any particular prokaryotic cells and, once such synthetic promoter sequences have been demonstrated, prepare constructs for which the electromagnetic radiation inducing the desired response(s) may be optimized.

The reactor assembly 1000 may be used with a combination of one or more eukaryotic and/or prokaryotic cells.

Referring again to FIG. 13, the reactor assembly 1000 is similar to the reactor 190 depicted in FIG. 11 but differs therefrom in that it additionally contains a hollow fiber reactor 1002. Hollow fiber biobreactors are well known to those skilled in the art. Reference may be had, e.g., to U.S. Pat. Nos. 4,889,812, 5,126,238, 5,616,421, 5,955,353, 6,001,585 (micro hollow fiber bioreactor), U.S. Pat. Nos. 6,207,448, 6,214,574, 5,512,480 (flow-through bioreactor with grooves for cell retention), and the like. The entire disclosure of each of these United States patents is hereby incorporated by reference into this specification.

Referring again to FIG. 13, a localized, time-varying electromagnetic field can be created at region 1004 by electromagnetic coil 196. As will be apparent, the coil 196 is wrapped around the periphery 1006 of hollow fiber bioreactor 1002.

In the embodiment depicted in FIG. 13, one or more secondary coils can be utilized that couple with the primary coil 196. By choosing the location and number of turns on these coils, one can selectively induce a desired amount of current that, in turn, will produce secondary localized magnetic fields in the localized areas, such as region 1004.

The region 1004 is surrounded by coils 1008 and 1010. In one embodiment, the coils 1008 and 1010 are variable inductors, whose degree of magnetic coupling may be varied.

Variable inductors assemblies, and means for varying their inductance, and well known to those skilled in the art. Reference may be had to U.S. Pat. Nos. 5,331,287, 5,239,239, 4,795,990, 6,340,831, and the like. The entire disclosure of each of these United States patents is hereby incorporated by reference into this specification.

Referring again to FIG. 13, the amount of current which flows through coils 1008 and 1010 may be varied by one of several means. Thus, e.g., controller 212 is preferably connected to a variable RL circuit comprised of coils 1008 or 1010 (or both); in such a circuit, when the resistance of the variable resistor is changed, the impedance of the circuit will change, the current that is induced in the circuit for any degree of magnetic coupling will change, and thus the magnetic field that is created by the inducted current also will change.

Thus, with this or a similar arrangement, one may impart selected magnetic fields at selected points in time (or continuously) to regions 1004.

Referring again to FIG. 13, the coil assembly 1014 is preferably comprised of a multiplicity of coaxial individual coils 1016. As will be apparent, as the number of such individual coils 1016 changes at any particular location 1012, e.g., the current induced in such a multiplicity of coils by coil 196 will vary, and the induced magnetic field in such location 1012 also will vary.

FIG. 14 is an enlarged view of a portion of section 1018 that illustrates how the reaction environment within any particular area of the bioreactor 1002 may be varied. Referring to FIG. 14, a partial sectional view of hollow fiber 1022.is shown As will be apparent, and in the embodiment depicted, fluid 1024 flows through hollow fiber 1022 in the direction of arrow 1026. As the temperature and/or the pressure and/or the flow rate and/or the composition of such fluid 1024 is varied, the conditions within hollow fiber 1022 also will vary.

The hollow fiber 1022 acts as a semi-permeable membrane. The cells 1028 being treated in the process of the invention generally are disposed on the periphery 1030 of the hollow fiber 1022. Reagents from the fluid 1024 diffuse in the direction of arrow 1032. Products formed by reactions of the cells 1028 tend to diffuse in the direction of arrows 1034. As will be apparent, the direction of diffusion is always from an area of greater concentration to an area of lesser concentration.

In the embodiment depicted in FIG. 14, a multiplicity of magnetizable particles 1036 are disposed on the periphery 1030 of the hollow fiber 1022. These particles 1036 are preferably paramagnetic, i.e, they are temporarily magnetizable but lose their concentration of magnetic flux lines upon the removal of the externally applied field.

Referring again to FIG. 13, the products 1041 produced by the cells within the bioreactor 1002 may be harvested in harvester 1040. by conventional means, and may harvested via port 1042. Reagents 1043 may be added to the system via hopper 1044. The temperature of material within the hopper 1044 and/or within the return line 1046 may be controlled, e.g., by the use of heating blankets 1048 and 1050.

FIG. 15 is a schematic view of a reactor assembly 1100 that, in one embodiment, has a capacity of about 1,000 liters. Referring to FIG. 15, reactor assembly 1100 is comprised reagent supply tank 1102 hydraulically connected to closed tubular reactor 1104. In one embodiment, tubular reactor 1104 has a diameter of from about 30 to about 40 centimeters and a length of from about 25 to about 35 diameters.

Referring again to FIG. 15, and in the preferred embodiment depicted therein, supply tank 1102 may be heated by heater 1106; thus, the temperature of the material in supply tank 1102 may be varied.

The flow rate from supply tank 1102 also may be varied with the use of variable speed pumps 1108 and 1110. As will be apparent, such flow rate can vary from 0 cubic centimeters per minute up to about 10,000 cubic centimeters per minute.

The material from pumps 1108 and 1110 may have flow rates which are the same, or which are different. Alternatively, or additionally, one reagent may be fed via pump 1108, and another reagent may be fed via line 1110. Other conventional hydraulic distribution arrangements also may be used.

The material fed from pumps 1108 and 1110 are fed to manifolds 1112 and 1114, respectively. These manifolds are adapted to effect the homogeneous dispersion of the material pumped therethrough into the reactor 1104.

Each of the manifolds 1112 and 1114 is comprised of a multiplicity of outlet ports 1116 and 1118, respectively. In the embodiment depicted, outlet ports 1116 and 1118 are disposed in the same direction and are substantially aligned with each other. In another embodiment, not shown, outlet ports 1116 and 1118 are disposed in different directions and/or are not aligned with each other.

A multiplicity of sensors 1120, 1122, and 1124 are disposed within the reactor 1104 and are adapted to sense the concentrations of one or more of the desired end products, of the desired intermediate products, of the enzymes and/or catalysts required, and/or the reagents required. This information is fed from the sensors 1120/1122/1124 to the controller 216. Thereafter, the controller 1126 can cause the power supply 1128 to generate one or more of the desired magnetic, electric, and/or electromagnetic fields that are optimum for producing whatever desired product, intermediate product, and/or enzyme is required for the continued high yield of the process. Alternatively, or additionally, additional amounts of such intermediate product and/or enzyme and/or reagent may be added to the system via line 1132.

In addition to monitoring the concentration of various catalysts and/or reagents, the controller 216 can also vary the feed rate of the feed materials, the temperature of the feed materials, the pressure of the feed materials (by varying the flow rate)

Referring again to FIG. 15, and in the preferred embodiment depicted therein, the reactor 1104 is comprised of a multiplicity of motionless mixers 1130 that which facilitate the mixing of the material flowing through the reactor without moving themselves. Reference may be had, e.g., to U.S. Pat Nos. 4,765,204, 4,643,584, 4,446,741, 5,397,180, and the like. The entire disclosure of each of these United States patents is hereby incorporated by reference into this specification.

Referring again to FIG. 15, it will be apparent that power supply 210, in response to signals from controller 216, can furnish various electric, magnetic, and/or electromagnetic fields to coil 196.

In the preferred embodiment depicted in FIG. 15, a impeller pump 1140 can selectively, intermittently, and/or continuously vary the mixing of and;/or the flow rate of the material within the reactor 1104. The desired product 1142 produced in the process may be continuously and/or intermittently harvested through port 1144.

FIG. 16 is a schematic of a reactor 1200 that is capable of generating multiple and/or different fields at different parts of the reactor.

Referring to FIG. 16, and in the preferred embodiment depicted therein, it will be seen that reactor assembly 1200 is comprised of a reagent supply tank 1102. In one embodiment, not shown, the line 1202 from supply tank 1102 is connected to a variable pump, such as variable pump 1108 (see FIG. 15).

One or more reagents may be supplied to the reagent supply tank 1102 via line 1204. Additionally, or alternatively, reagent may be added to the closed reactor tube 1206 via ports 1208 and/or 1210 and/or 1212. The latter process often is preferred in that it allows one to selectively add reagent at points in the process at which such reagent may be most advantageously used.

The intermediate and/or final product(s) 1214 may be removed from the reactor tube 1206 via port 1216. Alternatively, or additionally, one may discharge some or all of such product(s) via ports 1218, 1220, and/or 1222.

In the embodiment depicted in FIG. 16, a power supply 1224 is comprised of multiple outputs 1226, 1228, and 1230. These outputs may be the same, or they may be different, varying in type of energy, rate of energy, frequency, phase, amplitude, and the like.

The outputs 4226/1228/1230 are each connected to separate coils 1232, 1234, and 1236, respectively. Each of such coils 1232/1234/1236, when fed with a different energy pattern, will induce a different effect in areas 1238, 1240, and 1242 of the reactor tube 1206.

In the preferred embodiment depicted in FIG. 16, each of areas 1238, 1240, and 1242 is independently equipped with a heater 1244, 1246, and 1248, respectively. For the sake of simplicity or representation, the connections of these heating elements to a power supply (not shown) and/or to controller 216 have been omitted.

As will be apparent, in addition to controlling the electromagnetic energy with which each of the areas 1238, 1240, and/or 1242 are treated, one may also independently affect and control the temperature of each of such areas.

In the embodiment depicted in FIG. 16, an impeller pump 140 may facilitate circulation of fluid within the reactor 1206. The pump 1140 depicted in FIG. 16 is disposed near area 1234. One may also use additional impeller pumps 1140 disposed at area 1240 and/or 1242, and/or one may omit the use of impeller pump 1140 in area 1234 and add at least one such pump in one of the other areas 1240 and/or 1242.

Referring again to FIG. 16, one may vary the number of turns in coils 1232, 1234, and 1236 and/or the material such coils are made of and/or their physical dimensions and/or other properties so that, even if such coils are fed with the same electromagnetic energy, the will induce different effects.

FIG. 17 is a schematic of another reactor 1300 that is comprised of a closed hydraulic circuit 1302 comprised of conductive liquid 1304. The conductive liquid 1304 preferably has a conductivity of from about 0.5 to about 100 milliSiemens. In one embodiment, the conductive liquid 1304 has a conductivity of from about 5 to about 30 milliSiemens.

Referring again to FIG. 17, the conductive fluid 1304 preferably is comprised of a culture medium adapted to grow prokaryotic or eukaryotic cells. One may use one or more of the culture media disclosed in the prior art. Reference may be had, e.g., U.S. Pat. Nos. 6,245,555, 5,962,649, 6,210,911, 5,034,133, 5,567,807, 5,962,213, and the like. The entire disclosure of each of these United States patents is hereby incorporated by reference into this specification.

In one embodiment, the conductive fluid 1304 is comprised of both the culture medium and the cell or cells to be treated. The conductive fluid 1304, in the embodiment depicted, flows in the direction of arrow 1306, around conductive core 1308, in the direction of arrow 1310, and into treatment tank 1312.

In treatment tank 1312, the conductive fluid 1304 may be treated by any of the means heretofore described by reference to FIGS. 13, 14, 15, and 16. Thus, e.g., the conductive fluid may be heated by resistance heater 1244 (see FIG. 16). For the sake of simplicity of representation, the connections of said resistance heater to a power supply (not shown) and to controller 1314 have been omitted.

Thus, by way of further illustration, treatment tank 1312 preferably is comprised of variable pump 1108 so that the flow rate of the conductive fluid may be varied. For the sake of simplicity of representation, the connection of pump 1108 to controller 1314 has been omitted.

The treatment tank 1312 is comprised of at least one sensor 1316 disposed therein. This sensor is adapted to determine the type and amplitude of the current flowing in the conductive fluid. Depending upon the desired electromagnetic input to the cells in the conductive fluid, one can vary the output from the controller 1314 to produce the desired effects.

In the embodiment depicted in FIG. 17, controller 1314 is supplying an alternating current to the coil 1318. As used herein, the term alternating current includes any time-varying current such as, e.g., sine waves, square, triangular waves, and/or other complex wave forms. The type and intensity of the waveform(s) produced will depend upon the desired electric and/or magnetic signal one needs to induce within the conductive fluid 1304.

Referring again to FIG. 17, the alternating current flowing through coil 1318 will induce a magnetic field in transforming core 1320. It will change in intensity and direction as the alternating current flowing through coil 1318 changes.

The conductive fluid flows through a multiplicity of turns 1324, 1326, 1328, and 1330 disposed around the transforming core 1320. These turns act as a secondary on the transforming core, whereas the turns formed by coil 1318 act as a primary.

The alternating magnetic field in magnetic core 1320 will, in turn, induce an alternating current within the conductive fluid 1314. The current induced in the “secondary turns” will vary with the ratio of the turns on the primary to the turns on the secondary; and it will also vary with the voltage delivered to the primary.

Thus if one knows the current flowing in the conductive fluid 1304, one can vary such current by varying the voltage delivered by controller to conductive coil 1314. Similarly, one can affect the waveform of the induced current flowing in the secondary, the induced voltage flowing in the secondary, and other electrical properties.

As will be apparent to those skilled in the art, as the composition of the conductive fluid changes (by means of change in its type and concentration of protein), the electrical properties of the conductive fluid will change. This change can be compensated for.

Additionally, or alternatively, one may utilize one or more electrical circuits in the treatment tank 1314 which affect the inductance and/or capacitance and/or other properties of the current flowing in conductive fluid 1304. This circuit, not shown, preferably is operatively connected to the controller 1314.

The sensor 1316, or the sensors 1316 when more than one such sensor is used, is adapted to sense the type and amount of current and voltage in the conductive fluid, the conductivity of the conductive fluid, the concentration of various reagents in the conductive fluid, etc.

When the controller senses that a different electromagnetic signal should be applied to the conductive fluid, it can either vary the current and voltage fed to the conductive coil 1318, and/or it may vary the electrical circuit or circuits connected in series with the conductive fluid within treatment tank 1312.

When the controller senses that one or many essential reagents are either missing from or in low concentration within the conductive fluid 1304, it may cause such reagent(s) to be added via line 1322 and/or may cause suchreagent(s) to be produced via the process of this invention by means of utilization of the appropriate electromagnetic signals(s).

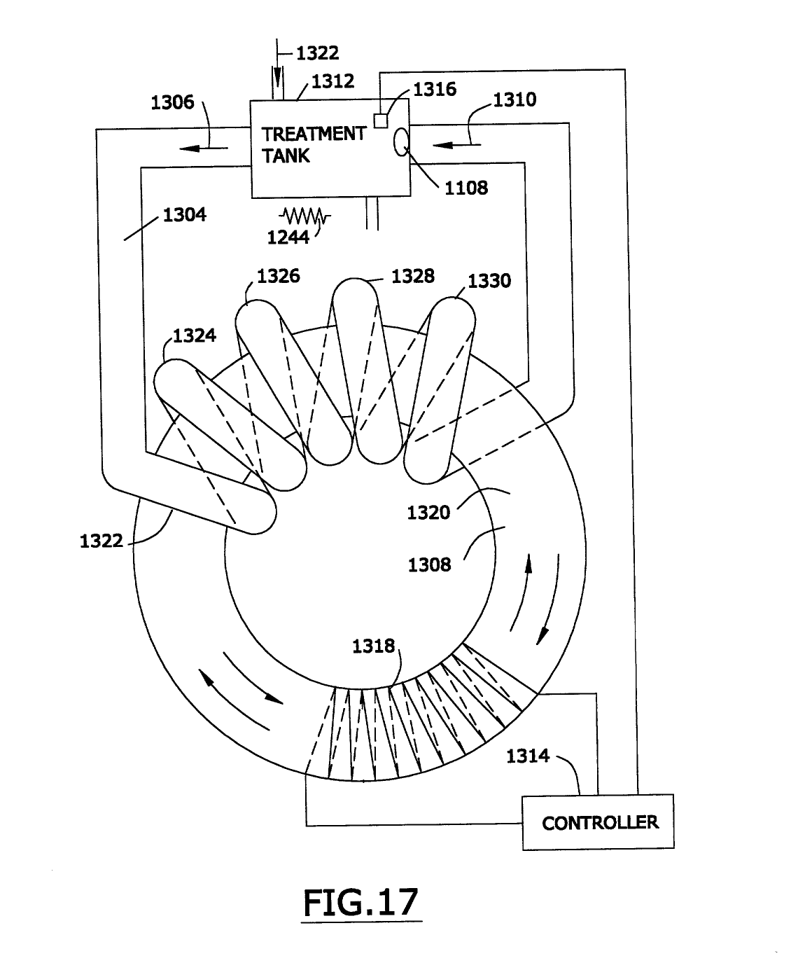

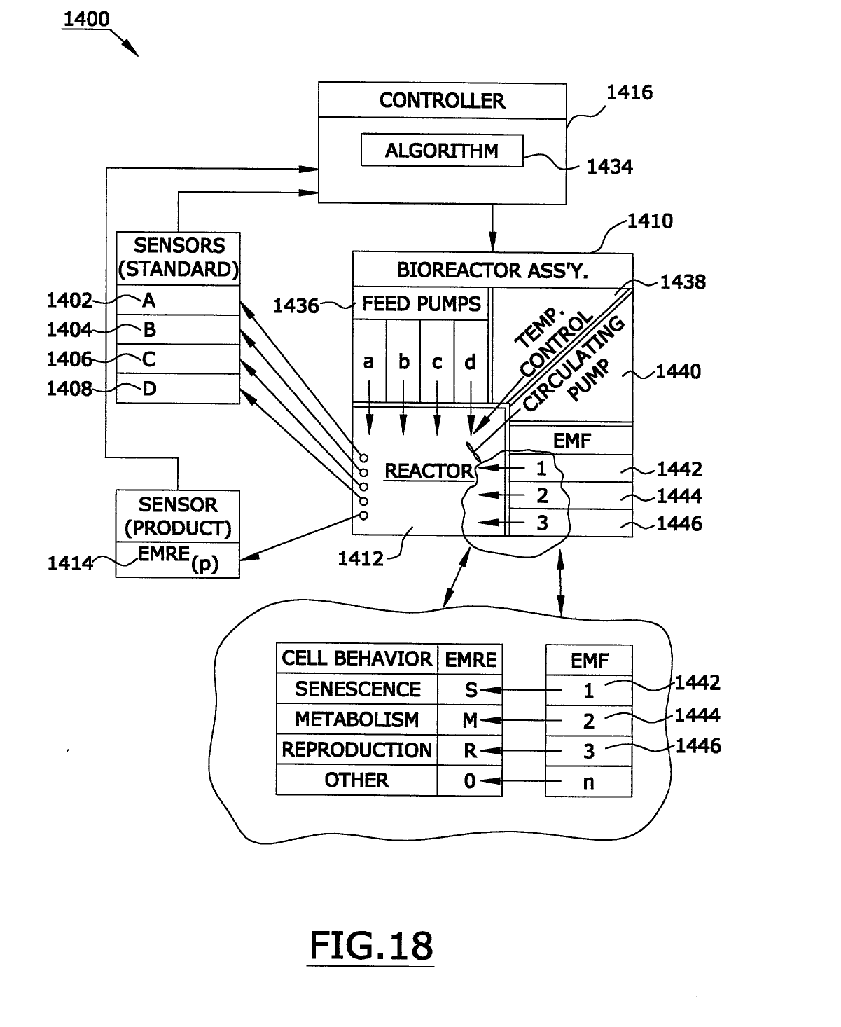

FIG. 18 is a schematic of a process 1400 for producing a desired biosynthetic product. In this process, a multiplicity of sensors 1402, 1404, 1406, and 1408 sense various properties of the biological system disposed within the reactor assembly 1410 and, in particular, within the reactor vessel 1412.

Referring again to FIG. 18, and in the preferred embodiment depicted therein, sensor 1414 is adapted to detect a reporter gene product, such as luciferase, produced in the reactor vessel 1412 as a result of the desired gene expression. In one embodiment of the process depicted in FIG. 18, an electromagnetic response element is linked to both a desired product and a “reporter product.” To the extent that the reporter product can be detected, its response is an indication of the response of and production of the desired product, inasmuch as both the reporter product and the desired product are linked to the same electromagnetic response element.

One may use luceriferase as the reporter product. Luciferase is the enzyme that functions in the bioluminescence reactions of the firefly; it catalyzes the oxidation of luciferin with the concomitant release of visible light.

Referring again to FIG. 18, when the reaction medium within the reactor vessel 1412 glows brightly, its photon output is proportional to the expression of the desired gene product.

Referring again to FIG. 18, one of the properties sensed by one or more of sensors 1402, 1404, 1406, and 1408 is the cell population within the reactor vessel 1412.

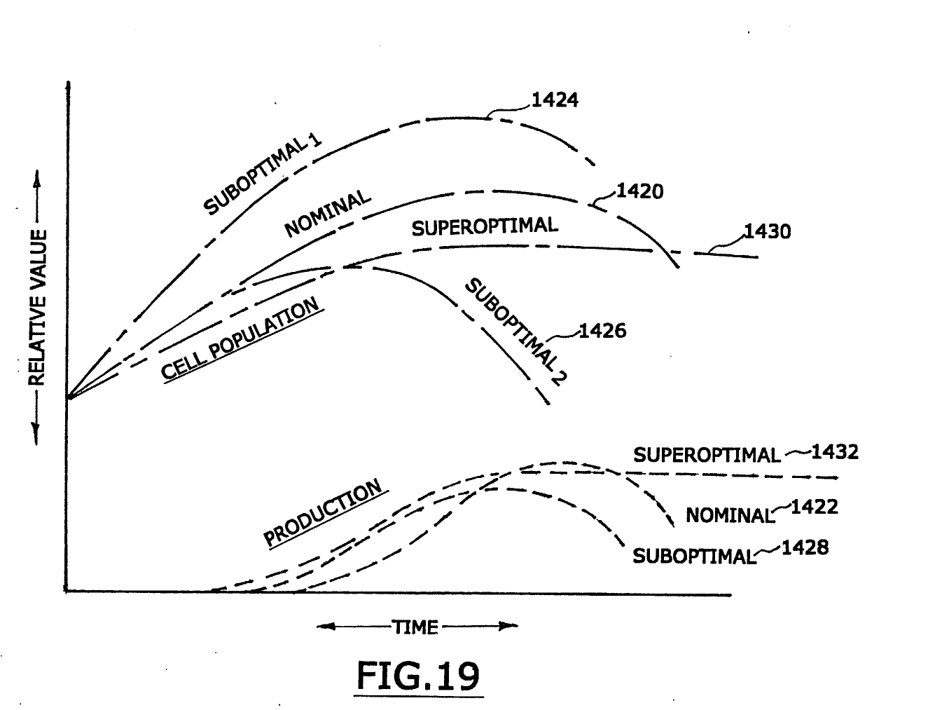

Referring to FIG. 19, the controller 1416 (see FIG. 18) can sense and plot the production of the desired product as a function of time. Some typical plots are shown in FIG. 19. These plots are representative of a general biosynthetic process and, thus are representative of batch mode processes, fed batch mode processes, and continuous mode processes, taking into consideration that the time axis should be expanded for fed-batch and for continuous bioreactor processes.

Referring to FIG. 19, the plot 1420 of the nominal cell population, and the plot 1422 of nominal production of the desired product, are representative of current practice. It should be noted that, with such current practice, after an initial rise in cell population of product production, there is a decrease.

Referring again to FIG. 19, plots 1424 and 1426 represent the suboptimal cell populations respectively caused by unregulated proliferation of cells, and cell death due to toxic conditions. Each of these suboptimal cell populations 1424 and 1426 results in suboptimal production of product, as shown in plot 1428.

Applicants' process utilizes appropriate electromagnetic response elements (EMRE's) that switch and/or modulate the factors in the cell life cycle such as, e.g., programmed cell death, apoptosis, cell division, general cell metabolism, expression of desired product, and/or expression of undesired by-products.

Thus, referring to FIG. 19, for any particular biological system the controller 1416 identifies a superoptimal cell population over time (see plot 1430 of FIG. 19) and a superoptimal product production over time (see plot 1432 of FIG. 19), and then it monitors the reaction vessel 1412 to determine any deviation from such superoptimal conditions.

The controller utilizes an algorithm 1434 to monitor the conditions within the reactor vessel 1412 and to adjust them, as necessary, to maintain the superoptimal conditions.

Such control may be effected by different means. One such means is to control the flow rate of one or more reagents into, through, or out of the reactor vessel 1412. This may be accomplished by means of feed pumps 1436. The feed pumps are preferably adapted to feed gaseous reagent, liquid reagent, solid reagent, and mixtures thereof.

One may utilize other control means known to those in the bioreactor art. Thus, by way of illustration and not limitation, one may effect such control by temperature control means 1438. Additionally, or alternatively, such control means may be effected by a circulation pump 1440.

In the process of the invention, it is preferred to effect such control, at least in part, by the use of one or more electromagnetic fields 1442, 1444, and 1446. Such electromagnetic fields may be the same; but it is preferred that they be different in that each electromagnetic response element has a particular electromagnetic signal which is optimal for it.

The electromagnetic fields 1442, 1444, and 1446 may be applied simultaneously, sequentially, or partially simultaneously and partially sequentially. These electromagnetic fields 1442, 1444, and 1446 may be used to increase the production of a desired intermediate and/or final product, and/or to decrease the production of a desired intermediate or a desired product (by the use of antisense transcripts). Alternatively, or additionally, one may use one or more negative regulatory control factors.

One or more of the electromagnetic fields 1442 et seq. may be chosen to prevent senescence. Senescence is programmed cell death and is also often referred to as apoptosis or preprogrammed cell death.

There are, however, anti-apoptosis genes. Reference may had, e.g., to U.S. Pat. Nos. 6,284,880, 6,340,673, 6,172,047, 6,346,389, 6,245,523 (survivin), U.S. Pat. Nos. 6,331,412, 6,077,709(survivin expression), U.S. Pat. Nos. 6,326,481, 6,228,603, 5,538,034, 6,414,134 (regulation of bcl-2 expression), and the like. The entire disclosure of each of these United States patents is hereby incorporated by reference into this specification. One or more of the electromagnetic fields 1442 et seq., in conjunction with the appropriate electromagnetic response element, may be used to produce such anti-apoptosis gene within the reactor vessel 1412.

In one embodiment, the anti-apoptosis product produced by the product of the instant invention in reactor vessel 1412 is telomerase. Reference may be had, e.g., to U.S. Pat. Nos. 6,010,846, 6,403,619, 6,337,065, 6,369,294, and the like. The entire disclosure of each of these United States patents is hereby incorporated by reference into this specification.

Referring again to FIG. 18, one or more of the electromagnetic fields 1442 et seq. may be used to regulate the metabolic rates of the cells within reactor vessel 1412. Thus, e.g., one may produce one or more of the factors that regulate the utilization of glucose and the production of adenosine triphosphate. Thus, e.g., one may cause the production of factors such as hexokinase, gluco-6-phospatase, phosgluoseisomerease, and the like. See, e.g. Chapter 7 (“Carbohydrate Metabolism 1: Major Metabolic Pathways and Their Control”) appearing at pages 291-358 Thomas M. Devlin's “Textbook of Biochemistry,” Third Edition (Wiley-Liss, New York, N.Y., 1992). Similarly, one may cause the production of enzymes that regulate the production of adenosine triphosphate from pyruvic acid, or one may decrease the production of such enzymes.

Referring again to FIG. 18, one may cause or minimize the production of enzymes or factors that facilitate cell reproduction, such, e.g., as those involved in the synthesis of DNA precursors; see, e.g., Chapter 13 of the aforementioned Devlin textbook. Thus, e.g., one may cause and/or inhibit the production of factors such as purines, pyrimidines that are the precursors for the production of DNA.

In applicant's U.S. Pat. No. 5,224,922, the entire disclosure of which is hereby incorporated by reference into this specification, there is disclosed and claimed A method for producing a regenerative effect in a biological system by exposing said biological system to an electromagnetic environment, said method comprising the steps of: generating a time-varying magnetic field; generating a static magnetic field in conjunction with said time-varying magnetic field in order to produce said regenerative effect.

The creation and manipulation of frequency/amplitude windows resulting from the application of electromagnetic fields timed to the appropriate most responsive state of a biological system is a means toward producing an optimized response. In the present invention, in one embodiment, the response is the optimal production of a polypeptide chain encoded by a nucleic acid sequence ligated downstream to an electromagnetic response element. Single-field and multi-field electromagnetic environments are optimally constructed to enhance the production of recombinant DNA proteins beyond that attainable by methods currently described in the art. For any method, the introduction of an electromagnetic response element and application of an electromagnetic field improves protein production.

Referring again to FIG. 1, and in the preferred embodiment depicted therein, in step 26 one or more host systems is exposed to a first electromagnetic radiation. This first electromagnetic radiation may be one type of such radiation, or a combination of two or more such types of radiation; and it may be applied simultaneously, or sequentially.

In step 28 of the process, the yield of the process is measured by conventional means. When the transfected biological system starts to become refractory, i.e., when the application of a specific electromagnetic radiation pattern ceases to increase the expression of the desired gene, then two alternatives are available.

In the first alternative, indicated as step 30, an electromagnetic radiation that is substantially different from the radiation initially applied to the system is used. Thus, e.g., one may change the amplitude of the radiation and/or its frequency component. Thus, e.g., one may apply a static field in addition to the time-varying field or instead of it.

In one embodiment, a change in the frequency or amplitude of as little as 10 percent is sufficient to cure the refractory state of the system.

Alternatively, and as is illustrated in step 32, one can apply electromagnetic noise to the biological system. while simultaneously applying no other electromagnetic energy thereto.

As used herein, the term electromagnetic noise refers to an electromagnetic radiation with certain characteristics. In the first place, the noise has bandwidth of frequencies of from about 2 to about 1,000 Hertz and, preferably, from about 3 to about 300 Hertz. In the second place, the noise has a root mean square (rms) amplitude of from about 0.01 to about 30 Gauss. In the third place, the noise is continuous. This noise is discussed, e.g., in a series of patents by T. A. Litovitz, including, e.g., U.S. Pat. Nos. 5,450,859 5,544,665, 5,566,685, 5,968,527, and 6,263,878. Reference also may be had to Litovitz's International Publication No. WO 98/37907 and to published patent application US 2001/0044643. The entire disclosure of each of these patents, patent applications, and international publications is hereby incorporated by reference in to this specification.

Referring again to FIG. 1, and in step 32 thereof, it is preferred to apply the electromagnetic noise to the transfected biological system for from about 1 to about 30 minutes. By comparison, in step 26, it is preferred to expose the system to electromagnetic radiation for from about 10 to about 30 minutes.

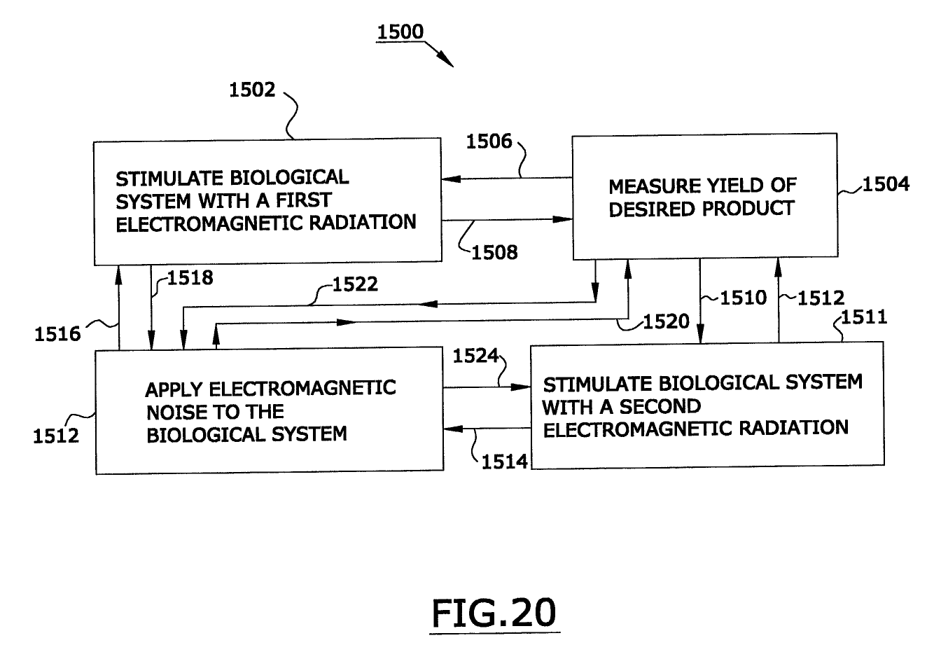

FIG. 20 is flow diagram illustrating one preferred process 1500 of the invention. Referring to FIG. 20, and in step 1502 thereof, a biological system is stimulated with a first electromagnetic radiation. In one embodiment, the radiation so used has a frequency of less than about 300 Hertz and an amplitude of less than about 30 Gauss.

It is preferred to expose the biological system to this radiation for from about 5 to about 60 minutes and, preferably, for from about 10 to about 30 minutes. Thereafter, in step 1504, the yield of the desired expression product is measured.

If the yield of the desired product is satisfactory, then one can repeat step 1502, as is schematically illustrated by line 1506. Thereafter, as is indicated by line 1508, one gain measures the yield of the desired product. This cycle is preferably repeated until the yield of the process starts to decline.

If the yield of the process begins to decline, and as indicated by line 1510, one may stimulate the biological system with a second, distinct electromagnetic radiation in step 1511. Thereafter, as indicated by line 1512, one may then measure the yield of the desired product.

Alternatively, or additionally, one may apply electromagnetic noise to the system (in step 1512) after the system has been stimulated with the second electromagnetic radiation in step 1511 (see line 1514).

Alternatively, or, additionally, one may apply electromagnetic noise to the biological system before or after it has been stimulated with the first electromagnetic radiation (see lines 1516 and 1518), and/or before or after one has measured the yield of desired product (se lines 1520 and 1522), and/or before or after it has been stimulated with the second electromagnetic radiation (see lines 1524 and 1514)

Other combinations and permutations of process steps will be apparent to those skilled in the art

In one embodiment, illustrated in step 34, after the application of noise in step 32 a second electromagnetic radiation, which may be the same as or different than the first electromagnetic radiation, is applied to the system.

In general, regardless of whether one is applying noise or electromagnetic radiation, it is preferred to apply such energy for from about 1 to about 60 minutes, and preferably from about 10 to about 30 minutes. The magnetic strength of the radiation generally should be from about 0.1 microTesla to about 1 Tesla. In one embodiment, it is preferred that the radiation have a magnetic strength less than about 3,000 microTeslas.

When using a time-varying magnetic field, it preferred to use a frequency of from about 1 to about 300 Hertz, in the extremely low frequency band. In one embodiment, this extremely low frequency component can be carried on a high frequency carrier, of the order of from 1 to about 200 Gigahertz.

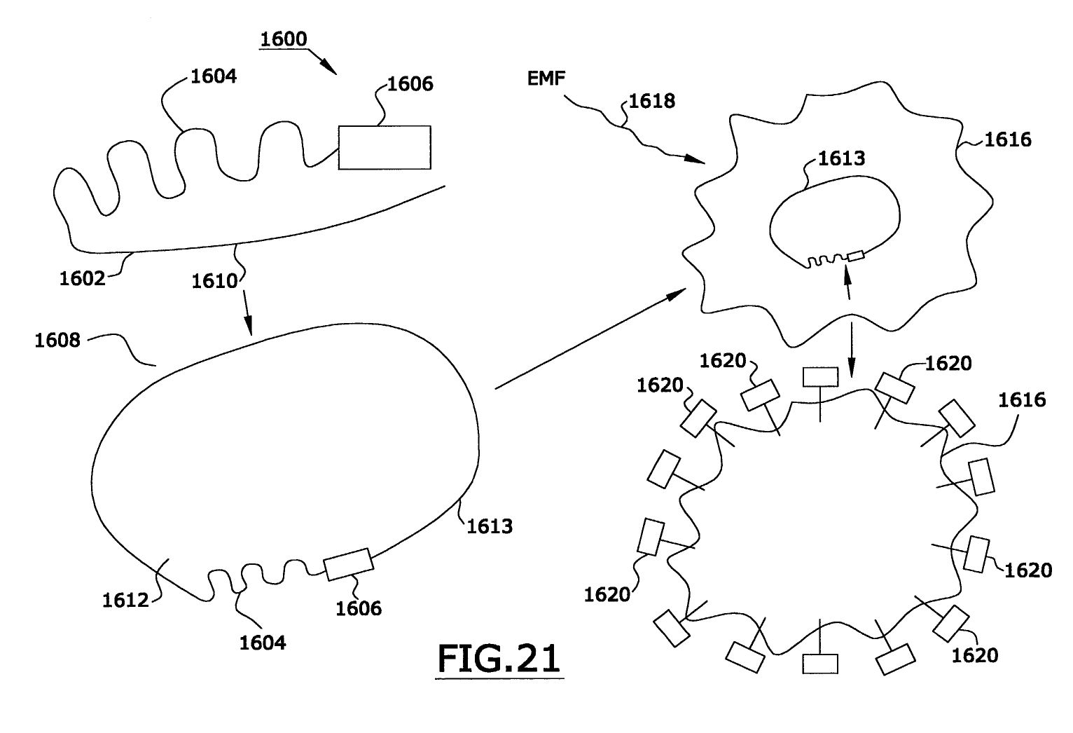

FIG. 21 is a schematic diagram of a process 1600 for preparing a DNA-based vaccine. In the first step of this process, step 1602, an electromagnetic response element (EMRE) 1604 is linked to a desired gene 1606. Gene 1606 preferably is one that codes for an antigenic agent against which the vaccine is made.

Thereafter, in step 1608, the DNA construct 1610 comprised of the gene 1606 and EMRE 1604 is integrated into a suitable DNA vector 1612.

In step 1614, the DNA vector is introduced into an living organism (not shown) wherein it is attached by and engulfed by a dendritic immune cell 1616.

Thereafter, in step 1618, the dendritic immune cell 1616 is contacted with electromagnetic radiation to cause the expression of gene 1606 to a higher degree than would normally be obtained.

The gene product of gene 1606, product 1620, are presented at the surface of the dendritic cell 1616. Because this product 1620 is a foreign protein, it will cause the production of an immune response against it, thereby causing the host organism to produce a vaccine against it.

Because the exposure to electromagnetic radiation ultimately causes more antigen be presented to the host's immune system, the immune response thus produced will be more robust than normally would produced.

In one embodiment, the process depicted in FIG. 21 is conducted ex vivo, and the dendritic cells with the foreign protein product are then introduced into the host.

In another embodiment, the dendritic cells with the foreign protein product are then exposed to additional electromagnetic radiation while residing in the host, to produce y et more foreign protein.

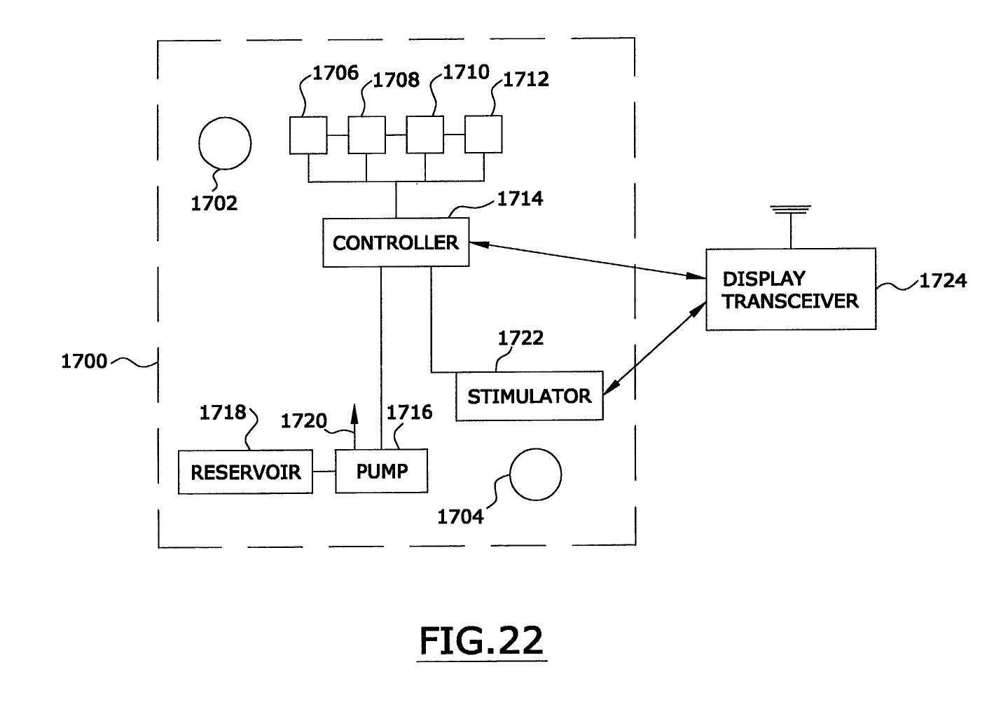

FIG. 22 is a schematic diagram of a biological system 1700 comprised of a first DNA construct 1702 comprising a first electromagnetic response element, and a second DNA construct 1704 comprising a second electromagnetic response element. Implantable sensors 1706, 1708, 1710, and 1712 communicate with both each other and controller 1714.

When controller 1714 becomes aware that a condition within biological system is less than ideal, it can take several actions.

In the first place, controller 1714 can cause pump 1716 to withdraw one or more desired products from reservoir 1718 and introduce such product(s) into biological system 1700 via line 1720.

In the second place, controller 1714 can cause stimulator 1722 to emit electromagnetic radiation towards' DNA constructs 1702 and/or 1704. This radiation may be nondirectional, or specifically aimed at the DNA constructs. It may be applied simultaneously and/or sequentially.

There need not necessarily be a direct link between the controller 1714 and the stimulator 1720. Telemetry links also may be used, in which case the stimulator 1722 preferably is comprised of a transceiver. In such a case, the stimulator/transceiver 1722 can also transmit information to a remote display 1724, which will display the status of the biological system 1700. In one aspect of this embodiment, the remote display 1724 also is comprised of a transceiver so that it can directly activate either the stimulator 1722 and/or the controller 1714.

The stimulator/transceiver 1722 can treat the organism 1700 in accordance with the processes of this invention, including the steps of intermittently subjecting such organism to electromagnetic radiation, and/or intermittently subjecting such organism to electromagnetic noise.

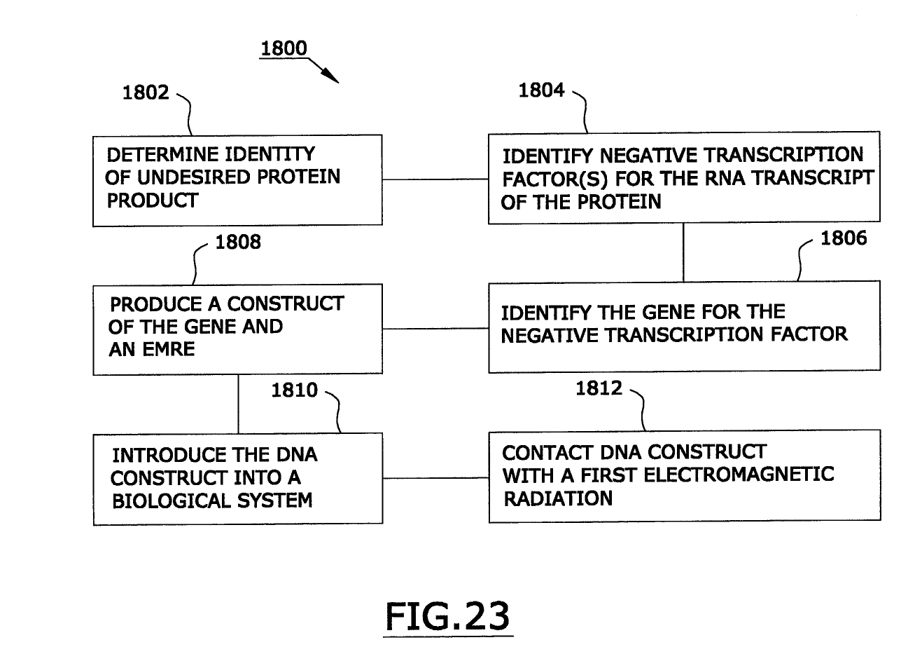

FIG. 23 is a flow diagram of a process 1800 for causing the cessation of expression of an undesired gene product, such as an RNA transcript or the protein product derived thereofrom.

In step 1802 of the process depicted in FIG. 23, the identity of the undesired protein product is determined. Thereafter, in step 1804, the identity of negative transcription factors that inhibit the formation of such protein is determined. The negative transcription factor may either inhibit the formation of the RNA transcript and/or of the protein derived from it.

Negative transcription factors are well known to those skilled in the art. Reference may be had, e.g., to U.S. Pat. Nos. 6,194,632, 6,368,829, 6,361,968, 6,303,756, 6,270,994, 6,251,628, 6,245,525, 6,200,800, 6,180,597, 6,171,857, 6,159,731, 6,103,869, 6,083,721, 5,972,643, 5,942,433, and the like. The entire disclosure of each of these United States patents is hereby incorporated by reference into this specification.

The negative transcription factor is generally a protein. Such negative transcription factor often binds to a promoter of a gene and effectively blocks such promoter from causing the expression of the gene.

The negative transcription factor can also be an artificially constructed antisense sequence that may either inhibit the transcription of the mRNA transcript, and/or the translation of the mRNA transcript.

For each negative transcription factor that inhibits the expression of a particular protein, the gene that expresses such negative tanscription factor is identified in step 1806.

Once the gene has been so identified, a construct of such gene and an electromagnetic response element is produced in step 1808.

The DNA construct produced in step 1808 is then introduced into a biological system in step 1810.

Thereafter, whenever it is desired to limit the production of the undesired gene product, the DNA construct is contacted with a first electromagnetic radiation in step 1812. This radiation causes the expression of the proteinaceous negative transcription factor that, in turn, limits the expression of the undesired protein.

In one embodiment, instead of producing a DNA construct comprised of a gene/promoter assembly for producing a proteinaceous negative transcription factor, one may produce a gene/promoter assembly for producing an antisense negative transcription factor that will inhibit either the transcription process or the translation process, as described above. This latter gene/promoter assembly also will preferably contain an electromagnetic response element (EMRE) that, upon exposure to electromagnetic radiation, will express an antisense mRNA transcript that, in turn, either blocks the transcription of the undesired gene or the translation of the undesired messenger RNA

In one embodiment, one may use a process analogous to creating conditional lethal mutations or other non-lethal conditional mutations to study the function of the protein that is inhibited by observing the biological consequences of inhibiting such protein production. This is analogous to studying conditional mutations in bacteria which are conditional upon changes in temperature.

The use of genes with conditional lethal mutations are well known. Reference may be had, e.g., to U.S. Pat. Nos. 6,207,407, 6,132,954, 5,976,821(identification of essential survival genes), U.S. Pat. Nos. 5,821,076, 5,756,305, 4,469,786, and the like. The entire disclosure of each of these United States patents is hereby incorporated by reference into this specification.

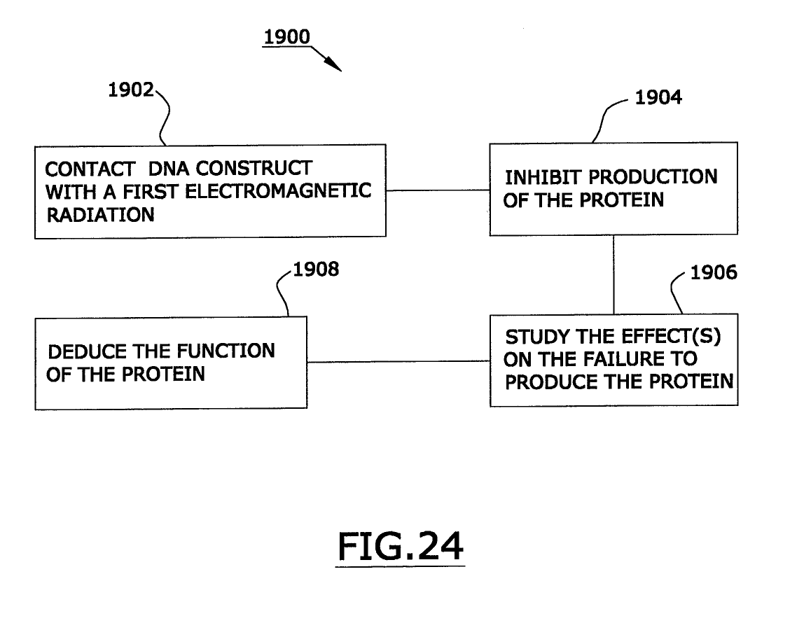

FIG. 24 illustrates a process 1900 that is analogous to processes described in the aforementioned patents. In step 1902 of the process, which is analogous to step 1812 of process 1800, a DNA construct that expresses a negative regulatory control factor and that is comprised of an electromagnetic response element is contacted with electromagnetic radiation to cause the expression of such negative regulatory control factor. It is preferred to generate a negative regulatory control factor that will inhibit expression of a specified gene.

The negative regulatory control factor whose expression is caused in step 1902 will, in step 1904, inhibit the expression of the protein being studied.

In step 1906, the result of the absence of the protein in question is studied. Thereafter, in step 1908, the function of the protein is deduced.

As will be apparent, the process of FIG. 24 is a means of creating conditional functional deficits in a biological system wherein the conditional element is electromagnetic radiation.

This process may be used in the creation of transgenic organisms to study functional deficits at the systemic level. It may also be used in gene therapy to downregulate the production of such factors as NF-kappaB that promote the malignant state in many cancers.

It is to be understood that the aforementioned description is illustrative only and that changes can be made in the apparatus, in the ingredients and their proportions, and in the sequence of combinations and process steps, as well as in other aspects of the invention discussed herein, without departing from the scope of the invention as defined in the following claims.

We claim:

1. A process for stimulating the expression of a gene comprising the steps of disposing a first electromagnetic responsive DNA construct in a biological system, disposing a second electromagnetic responsive DNA construct in such biological system, stimulating said first electromagnetic responsive DNA construct with electromagnetic energy, stimulating said second electromagnetic responsive DNA construct with electromagnetic energy, expressing a first RNA transcript from said first electromagnetic responsive DNA construct, and expressing a second RNA transcript from said second electromagnetic responsive DNA construct.

2. A biological system comprised of a first electromagnetic responsive gene product, a second electromagnetic responsive gene product, means for stimulating said first electromagnetic responsive gene product with a electromagnetic energy, means for stimulating said second electromagnetic responsive gene product with electromagnetic energy, means for expressing a first RNA transcript with said first electromagnetic responsive gene product, and means for expressing a second RNA transcript with said second electromagnetic responsive gene product.

3. A bioreactor comprised of a biological system, wherein said biological system is comprised of a first electromagnetic responsive DNA construct comprised of a electromagnetic response element ligated to a first polynucleotide sequence, means for stimulating said first electromagnetic response DNA construct with electromagnetic energy, and means for expressing a first RNA transcript from said first electromagnetic response DNA constructt, wherein said bioreactor is comprised of a sensor for sensing a property of said biological system and means for generating an electromagnetic signal in response to information received from said sensor.

4. The bioreactor as recited in claim 3, wherein said bioreactor is comprised of a first electromagnetic coil assembly.

5. The bioreactor as recited in claim 4, wherein said bioreactor is comprised of a controller operatively connected to said first electromagnetic coil assembly.

6. The bioreactor as recited in claim 5, wherein said sensor is operatively connected to said controller.

7. The bioreactor as recited in claim 6, wherein said bioreactor is comprised of a power supply connected to said first electromagnetic coil assembly.

8. The bioreactor as recited in claim 7, wherein said bioreactor further comprises a exit line.

9. The bioreactor as recited in claim 8, wherein said electromagnetic coil is disposed around said exit line.

10. The bioreactor as recited in claim 7, further comprising a reactor vessel comprised of hollow fibers.

11. The bioreactor as recited in claim 10, wherein said hollow fibers are disposed within a hollow fiber bioreactor comprised of a multiplicity said hollow fibers on whose outer surface a bonded a multiplicity of cells.

12. The bioreactor as recited in claim 11, wherein said electromagnetic coil assembly is disposed around said hollow fiber bioreactor.

13. The bioreactor as recited in claim 12, further a second electromagnetic coil assembly disposed around a multiplicity of hollow fibers.

14. The bioreactor as recited in claim 13, wherein said second electromagnetic coil assembly is comprised of a continuous electromagnetic coil.

15. The bioreactor as recited in claim 13, wherein said second electromagnetic coil assembly is comprised of a multiplicity of individual closed coils that are adjacent to each other but not contiguous with each other.

16. The bioreactor as recited in claim 12, wherein a multiplicity of magnetic particles are disposed on said hollow fibers.

17. The bioreactor as recited in claim 16, wherein a multiplicity of cells are disposed on said hollow fibers.

18. The bioreactor as recited in claim 11, further comprising means for delivering a first electromagnetic signal to a first region of said hollow fiber bioreactor.

19. The bioreactor as recited in claim 18, further comprising means for delivering a second electromagnetic signal to a second region of said hollow fiber bioreactor.

20. The bioreactor as recited in claim 19, further comprising means for delivering a third electromagnetic signal to a third region of said hollow fiber bioreactor.

21. The bioreactor as recited in claim 7, wherein said bioreactor is comprised of means for circulating a cell culture within said bioreactor.

22. The bioreactor as recited in claim 21, wherein said means for circulating said cell culture is comprised of a pump.

22. The bioreactor as recited in claim 21, wherein said pump is a variable pump.

23. The bioreactor as recited in claim 22, wherein said bioreactor is comprised of a mean a in-line mixer.

24. The bioreactor as recited in claim 23, wherein said bioreactor is comprised of a multiplicity of said in-line mixers.

25. The bioreactor as recited in claim 24, wherein said bioreactor is comprised of a multiplicity of sensors.

26. The bioreactor as recited in claim 25, wherein said bioreactor is comprised of a multiplicity of input ports.

27. The bioreactor as recited in claim 7, further comprising a second electromagnetic coil assembly connected to said power supply.

28. The bioreactor as recited in claim 7, further comprising a third electromagnetic coil assembly connected to said power supply.

29. The bioreactor as recited in claim 28, wherein said bioreactor is comprised of a closed loop.

30. The bioreactor as recited in claim 29, wherein said first electromagnetic coil is disposed around a first section of said closed loop.

31. The bioreactor as recited in claim 30, wherein said second electromagnetic coil is disposed around a second section of said closed loop.

32. The bioreactor as recited in claim 31, wherein said third electromagnetic coil is disposed around said third section of said closed loop.

33. The bioreactor as recited in claim 7, wherein said bioreactor is comprised of a closed loop.

34. The bioreactor as recited in claim 33, wherein said closed loop is comprised of conductive fluid.

35. The bioreactor as recited in claim 34, wherein said conductive fluid has a conductivity of from about 0.5 to about 100 milliSiemens.

36. The bioreactor as recited in claim 35, wherein said conductive fluid has a conductivity of from about 5 to about 30 milliSiemens.

37. The bioreactor as recited in claim 36, further comprising means for circulating said conductive fluid within said closed loop.

38. The bioreactor as recited in claim 39, wherein said closed loop is disposed around a transformer core.

39. The bioreactor as recited in claim 38, wherein said transformer core is a torroidal transformer core.

40. The bioreactor as recited in claim 39, wherein said first electromagnetic oil is disposed around said torroidal transformer core.

41. The bioreactor as recited in claim 40, further comprising means for sensing a property of said conductive fluid.

42. The bioreactor as recited in claim 42, further comprising a controller connected to said means for sensing a property of said conductive fluid.

43. The bioreactor as recited in claim 7, wherein said bioreactor is comprised of a biological system comprising an electromagnetic response element linked to a first gene that expresses a product, an electromagnetic response element linked to a second gene that expresses a regulatory control factor, and an electromagnetic response element linked to a third gene that expresses an anti-apototic

44. A biological system comprised of a first DNA construct comprised of a first electromagnetic response element ligated to a first polynucleotide sequence and a second DNA construct comprised of a second electromagnetic response element ligated to a second polynucleotide sequence, wherein said first DNA construct expresses a positive regulatory control element in response to exposure to electromagnetic radiation, and wherein said second DNA construct expresses a negative regulatory control element in response to electromagnetic radiation.

45. The biological system as recited in claim 44, wherein said biological system is comprised of implantable means for emitting said electromagnetic radiation.

46. The biological system as recited in claim 45, wherein said biological system is comprised of an implantable sensor.

47. The biological system as recited in claim 46, wherein said biological system is comprised of an implantable pump.

48. A process for regulating a biological system, comprising the steps of:
(a) disposing within said biological system a first electromagnetic response element ligated to a first polynucleotide sequence and a second DNA construct comprised of a second electromagnetic response element ligated to a second polynucleotide sequence, wherein said first DNA construct expresses a positive regulatory control element in response to exposure to electromagnetic radiation, and wherein said second DNA construct expresses a negative regulatory control element in response to electromagnetic radiation,
(b) exposing said first DNA construct to electromagnetic radiation, and
(c) exposing said second DNA construct to electromagnetic radiation.

49. The process as recited in claim 48, wherein said biological system is exposed to electromagnetic noise.

50. A biological system comprised of a first DNA construct comprised of a first electromagnetic response element ligated to a first polynucleotide sequence, wherein said first DNA construct expresses a negative regulatory control element in response to exposure to electromagnetic radiation.
